(19) United States
(12) Patent Application Publication
(10) Pub. No.: US 2010/0028735 A1
(43) Pub. Date: Feb. 4, 2010

(54) PROCESS FOR CONVERTING METHANE INTO A HIGHER ALKANE MIXTURE

(75) Inventors: Jean Marie Basset, Caluire (FR); Sudhakar Chakka, Naperville, IL (US); Daravong Soulivong, Lyon (FR); Mostafa Taoufik, Villeurbanne (FR); Jean Thivolle-Cazat, Fontaines sur Saone (FR)

Correspondence Address: NIXON & VANDERHYE, PC, 901 NORTH GLEBE ROAD, 11TH FLOOR, ARLINGTON, VA, 22203, US

(73) Assignee: BP OIL INTERNATIONAL LIMITED

(21) Appl. No.: 12/448,275

(22) PCT Filed: Dec. 18, 2007
(86) PCT No.: PCT/GB2007/004866

§ 371 Date: Jun. 16, 2009

(30) Foreign Application Priority Data
Dec. 19, 2006 (EP) 06256432.3
Dec. 19, 2006 (EP) 06256433.1
Dec. 19, 2006 (EP) 06256457.0

(51) Int. Cl. H01M 8/04; C07C 2/76; C01B 3/02

(52) U.S. Cl. 429/17; 585/310; 585/316; 423/648.1

(57) ABSTRACT

The present invention relates to a process for converting methane into a higher alkane mixture, preferably a liquid alkane mixture, comprising (C₃₊) alkanes having 3 carbon atoms and more, preferably (C₄₊) alkanes having 4 carbon atoms, especially (C₅₊) alkanes having 5 carbon atoms and more. The process comprises:—a stage (1) comprising contacting methane with a metal catalyst (C1) capable of producing, in contact with alkane, reactions involving the splitting and recombining of C—C and/or C—H and/or C-metal bonds, so as to form by a non-oxidative methane coupling reaction a mixture (M1) comprising ethane and hydrogen,—preferably a stage (2) comprising fractionating the mixture (M1) so as to separate and to isolate the ethane, and—a stage (3) comprising, preferably simultaneously, (i) contacting the mixture (M1) or preferably the ethane isolated in stage (2) with a metal catalyst (C2) capable of producing, in contact with alkane, reactions involving the splitting and recombining of C—C and/or C—H and/or C-metal bonds, said catalyst being identical to or different from catalyst (C1), so as to form by simultaneous ethane self- and cross-metathesis reactions a mixture (M2) comprising methane and the higher alkane mixture, and (ii) fractionating the mixture (M2) so as to separate and to isolate said higher alkane mixture, preferably in liquid form, and particularly the methane which is preferably recycled into stage (1). The hydrogen produced in stage (1) is preferably separated and isolated in stage (2) and then used for various applications and stages, e.g. for producing thermal and/or electrical energies preferably employed to run the process.

The present invention relates to a process for converting methane into a higher alkane mixture.

Methane is present in very large amounts as natural gas which is widely distributed in the world. Natural gas deposit sites are often very far removed from one another and from large markets or consumption areas, such as in Alaska or in Siberia. It follows that methane and generally natural gas from which methane can be derived, often have to be transported over long distances. It is however difficult to transport them because of their gaseous nature. Methane or natural gas from which methane can be generally derived, can nevertheless be liquefied at very low temperature and under high pressure, and thus be transported more easily in liquid form. However, the energy and capital costs in achieving the liquefaction conditions and then in transporting the methane or the natural gas in liquid form under these conditions are very high and can be often prohibitive as regards to the exploitation of natural gas deposits, particularly ones of relatively low capacity.

A solution would be to convert at low costs the methane or the natural gas from which methane can be derived, into mixtures of higher alkanes which would be liquid under standard temperature and pressure conditions or conditions relatively close to the latter, and which could therefore be easily transportable in liquid form. Such a conversion process would have to be simple and realisable at relatively low costs, enabling it to be installed directly on the natural gas deposit sites. This is the solution which the present invention proposes.

Processes for converting methane into liquid fuels by an oxidizing route have already been known for a long time. The processes comprise first of all a reforming stage for converting methane into a mixture of carbon monoxide and hydrogen, also called “synthesis gas” or “syngas”, and then one or more stages for converting said mixture into diesel and other easily transportable fuels. However, said processes require a great deal of energy and high investment in particular in order to achieve very high temperatures which generally can exceed 800° C. These processes, in addition to being very expensive to build and to operate, also generate substantial quantities of green house causing carbon dioxide. Furthermore, the “carbon atom efficiency” of such processes is undesirably low.

International Patent Application WO 98/02244 discloses a process for converting C, alkanes into their higher and lower homologues. According to the process, one or more starting alkanes are reacted on a metal catalyst suitable for alkane metathesis and chosen in particular from metal hydrides and organometallic compounds fixed to a solid support. However, the process does not relate to conversion of methane or natural gas, but to conversion of alkanes having at least 2 carbon atoms. Various conversions are shown as examples, in particular those of ethane, propane, butane or isobutane respectively into their higher and lower homologues.

International Patent Applications WO 01/04077 and WO 03/066552 disclose processes for preparing alkanes by reacting methane with at least one other starting alkane in the presence of a metal catalyst suitable for producing, in contact with alkane, reactions involving the splitting and recombining of carbon-carbon and/or carbon-hydrogen and/or carbon-metal bonds. Said reactions, known under the term “methane-olysis” reactions, permit the preparation of at least one or two alkanes having a number of carbon atoms less than or equal to that of the starting alkane and at least equal to 2. Various methane-olysis reactions are shown as examples, in particular by reacting methane with ethane, methane with propane or methane with n-butane. It appears that methane-olysis of ethane leads to formation of methane and ethane, that is to say to formation of the starting products of the reaction. In addition, it is shown that methane-olysis of propane leads to formation of ethane, and that of n-butane permits ethane and propane to be prepared. Thus, it is taught that such a methane-olysis process leads to producing alkanes whose number of carbon atoms is not higher than that of starting alkane, and therefore could not be suitable for producing higher alkane mixtures.

International Patent Applications WO 03/104171 discloses a process for converting methane into ethane by contacting methane with a metal catalyst chosen in particular from metal hydrides, metal organic compounds and mixtures thereof. The process employs a methane coupling reaction, also called a methane homologation reaction, which essentially produces a mixture of ethane and hydrogen. It is suggested that the ethane thus prepared can then be used in other processes for upgrading of the ethane, such as dehydrogenation, catalytic cracking or thermal cracking, in order to prepare olefins, such as ethylene. International Patent Application WO 03/104171 neither envisages a process for the preparation of mixtures of higher alkanes beyond ethane, nor suggests that ethane is the key point for transforming methane into mixtures of higher alkanes which would be liquid under standard temperature and pressure conditions or conditions close to the latter.

International Patent Application WO 2006/060692 discloses supported metal clusters comprising one or more metals of Groups 4, 5 and/or 6 of the Periodic Table of the Elements, and which can be used as catalysts for alkane reactions. More particularly, the supported metal clusters are capable of being used to catalyze the metathesis (also referred to as disproportionation) of alkanes, conversions of alkanes with each other to give alkane products with molecular weights different from those of the starting alkanes, and conversion of methane to higher molecular-weight alkanes. The only alkane reactions described and shown as examples relate to a self-metathesis of ethane to give propane and methane, and a conversion of methane and n-butane (i.e. methane-olysis of n-butane) to give propane and ethane. However, the Application neither discloses, nor suggests a process for converting methane into mixtures of higher alkanes which would be liquid under standard temperature and pressure conditions or conditions close to the latter. Indeed, it is taught that methane reacting with other starting alkane (e.g. n-butane) leads to formation of final alkanes (e.g. propane and ethane) with molecular weight lower than that of the starting alkane, and therefore could not be suitable for producing higher alkane mixtures.

Thus, it appears that none of the above-mentioned patent applications proposes, nor suggests a process for converting methane or natural gas from which methane can be derived, into a higher alkane mixture, in particular a liquid alkane mixture, such as a mixture of alkanes which is liquid under standard temperature and pressure conditions or conditions close to the latter, and capable more particularly of being easily transportable in liquid form under such conditions.

A process has now been found for converting methane into a higher alkane mixture, in particular a liquid alkane mixture, such as a mixture comprising alkanes which is liquid under standard temperature and pressure conditions or conditions close to the latter. The process has the advantage of being able to produce from methane, a higher alkane mixture, in particular a liquid alkane mixture, e.g. a mixture of (C₃₊) alkanes having 3 carbon atoms and more (e.g. a mixture of C₃ to C₁₅, C₃ to C₁₃, C₃ to C₁₀, or C₃ to C₈ alkanes), or preferably a mixture of (C₄₊) alkanes having 4 carbon atoms and more (e.g. a mixture of C₄ to C₁₅, C₄ to C₁₃, C₄ to C₁₀, or C₄ to C₈ alkanes), or especially a mixture of (C₅₊) alkanes having 5 carbon atoms and more (e.g. a mixture of C₅ to C₁₅, C₅ to C₁₃, C₅ to C₁₀, or C₅ to C₈ alkanes). The present process also has the advantage of being able to produce a higher alkane mixture, preferably a liquid alkane mixture, with a high degree of purity, in particular completely free of alkenes, alkynes, aromatic compounds, carbon monoxide, carbon dioxide, sulphur- or nitrogen-containing products. It does not generate waste products and particularly no carbon dioxide, and thus can be considered as a clean process for the environment. It also has the advantage of being able to produce a higher alkane mixture, preferably a liquid mixture of alkanes having a narrow distribution of the number of carbon atoms, in particular ranging for example from 3 to 15 or preferably from 4 to 15, particularly from 3 to 13 or preferably from 4 to 13, more particularly from 3 to 10 or preferably from 4 to 10, or even from 3 to 8 or preferably from 4 to 8 carbon atoms. The higher alkane mixture thus obtained can be easily processed into high value fuels and chemicals in refineries. The present invention has, in addition, the advantage of proposing a process by a non-oxidizing route, of being simple and direct, and consequently of being relatively inexpensive. Indeed, the present process generally requires significantly lower capital and operating costs in comparison with the known processes, because the reactions involved in the present process take place at moderate temperatures and pressures, and substantial amounts of hydrogen are produced as a by-product which can supply all the energy needs for the present process. In addition, the present process may require very small amounts of cryogenic supplying and does not require the need of oxygen. Furthermore, one of the most important advantages of the process of the present invention relates to its potential for an extremely high “carbon atom efficiency” which can be of 95%, or even of 99% or close to 100% for converting methane or natural gas (from which methane can be derived) into a higher alkane mixture, preferably a liquid alkane mixture which can be easily transportable. Generally, “carbon atom efficiency” is presently defined as the % of carbon atoms of the starting methane or natural gas (from which methane can be derived) ending up in the final higher alkane mixture, preferably the liquid alkane mixture.

More particularly, the present invention relates to a process for converting methane into a higher alkane mixture, preferably a liquid alkane mixture, comprising (C₃₊) alkanes having 3 carbon atoms and more, preferably (C₄₊) alkanes having 4 carbon atoms and more, especially (C₅₊) alkanes having 5 carbon atoms and more, characterized in that it comprises the following stages:

a stage (1) comprising contacting methane with a metal catalyst (C1) capable of producing, in contact with alkane, reactions involving the splitting and recombining of carbon-carbon and/or carbon-hydrogen and/or carbon-metal bonds, so as to form by a non-oxidative methane coupling reaction a mixture (M1) comprising ethane and hydrogen,

preferably a stage (2) comprising fractionating the mixture (M1), so as to separate and to isolate the ethane, and

a stage (3) comprising, preferably simultaneously:
(i) contacting the mixture (M1) obtained in stage (1) or preferably the ethane isolated in stage (2) with a metal catalyst (C2) capable of producing, in contact with alkane, reactions involving the splitting and recombining of carbon-carbon and/or carbon-hydrogen and/or carbon-metal bonds, said catalyst being identical to or different from the catalyst (C1), so as to form by simultaneous ethane self- and cross-metathesis reactions a mixture (M2) comprising methane and the higher alkane mixture comprising the (C₃₊), preferably the (C₄₊), especially the (C₅₊) alkanes, and
(ii) fractionating the mixture (M2) so as to separate and to isolate the higher alkane mixture comprising the (C₃₊), preferably the (C₄₊), especially the (C₅₊) alkanes, preferably in liquid form, and particularly to separate and to isolate the methane which is recycled into stage (1).

The methane used in stage (1) or the natural gas which can substitute the methane or from which the methane can be derived and used in stage (1), may comprise one or more poisons capable particularly of deactivating the metal catalysts used in the present process, and optionally one or more impurities, such as nitrogen (N₂). Thus, the process advantageously may comprise, prior to stage (1), a purification stage comprising removing from the methane or the natural gas, one or more poisons particularly capable of deactivating the metal catalysts (C1) and/or (C2) as employed in the process, and/or one or more impurities, such as nitrogen (N₂). The purification stage can comprise removing one or more poisons particularly chosen from water, carbon dioxide and sulphur compounds especially selected from hydrogen sulphide, carbonyl sulphide and mercaptans, and/or one or more impurities, such as nitrogen (N₂). The purification stage can be performed in various ways, either intermittently (or discontinuously), or preferably continuously.

Several natural gas deposits may comprise substantially high concentrations of nitrogen (N₂) as impurity. Since generally nitrogen (N₂) is not anticipated to harm the metal catalysts (C1) and/or (C2) as employed in the process, it might not be necessary to remove the nitrogen (N₂) from-the methane or the natural gas prior to contacting said methane or natural gas with the metal catalyst (C1) in stage (1) of the process, particularly if the concentration of nitrogen (N₂) is relatively small, e.g. lower than 1% by volume of the total volume of the gas. However, it is advisable to remove nitrogen (N₂) from the methane or the natural gas prior to contacting said methane or natural gas with the metal catalyst (C1) in stage (1).

In the purification stage, water can be removed by subjecting the methane or the natural gas to a dehydration process preferably chosen from cryogenic dehydration, dehydration by absorption and adsorptive dehydration. Generally, a cryogenic dehydration may comprise cooling the methane or the natural gas until the components to be removed precipitate by condensation or formation of hydrates. Methanol, glycol or a paraffin solvent can be added to the methane or the natural gas and used during such a cryogenic dehydration. The cooling of the methane or the natural gas can be achieved by the Joule-Thomson expansion.

Generally, the dehydration by absorption of the methane or the natural gas can comprise a glycol absorption wherein the glycol, such as mono-, di- or preferably tri-ethylene glycol, exhibits a high absorption capacity for water.

Generally, the adsorptive dehydration of the methane or the natural gas may comprise contacting said methane or natural gas with any adsorbent suitable for retaining water, preferably chosen from molecular sieves, silica gels and Na₂O-containing silica.

In the purification stage, sulphur compounds and carbon dioxide can be removed from the methane or the natural gas by any process preferably chosen from physical absorption processes, chemical absorption processes, physical-chemical absorption processes, liquid oxidation processes, adsorption processes and membrane processes.

Generally, the absorption processes may comprise scrubbing the methane or the natural gas components with the help of a solvent which is reversibly bound by chemical and/or physical absorption to said methane or natural gas components in a scrubber. The absorption processes may also comprise a regeneration step wherein the methane or the natural gas components are then desorbed unchanged, and then the solvent can be recovered and recycled to the scrubber. Generally, the physical absorption processes may comprise physically dissolving carbon dioxide and hydrogen sulphide in a solvent preferably chosen from N-methylpyrrolidone, a mixture of ploy(ethylene glycol dimethyl ether), poly(ethylene glycol methyl isopropyl ether) and propylene carbonate. Generally, the chemical absorption processes may comprise scrubbing the methane or the natural gas with the help of an aqueous solution of an alkanolamine used as an absorbent and preferably chosen from monoethanolamine, diethanolamine, diisopropylamine, diglycolamine and methyldiethanolamine. Generally, the physical-chemical absorption processes may comprise a combination of solvents as previously described in the physical absorption processes and in the chemical absorption processes.

In the purification stage, the liquid oxidation processes generally comprises the following steps: (i) absorption of hydrogen sulphide in an alkaline solution, e.g. a solution of NaHCO₃—Na₂CO₃, (ii) oxidation of the dissolved hydrogen sulphide ions preferably by vanadates or iron(III)aminopolycarboxylic acid chelates, to elemental sulphur simultaneously with reduction of a chemical oxygen carrier, e.g. anthraquinonedisulphonic acids, (iii) reoxidation of the active components with air, and (iv) separation of the elemental sulphur by filtration, centrifugation, flotation or sedimentation.

In the purification stage, the adsorption processes are generally used for removing hydrogen sulphide and carbon dioxide from the methane or the natural gas, and may comprise contacting said methane or natural gas with any adsorbent preferably chosen from activated charcoal, iron oxide, zinc oxide and zeolite molecular sieves.

In the purification stage, the membrane processes especially used for natural gas can be simultaneously performed for separation of carbon dioxide and methane, removal of carbon dioxide and hydrogen sulphide, dehydration and recovery of methane and optionally the rest of the natural gas essentially comprising a mixture of (C₂₊) alkanes having 2 carbon atoms and more, preferably a mixture of C₂ to C₆ alkanes (hereafter referred as the (C₂₋₆) alkanes) or a mixture of higher alkanes. Generally, the membrane processes may comprise the following steps: (i) absorption from the gas phase into the membrane matrix, (ii) diffusion through the membrane, and (iii) desorption out of the membrane into the gas phase. The membranes generally used are polymer membranes preferably chosen from asymmetric cellulose acetate or triacetate, composite layers silicone/polysulphone, composite layers polyetherimide and composite layers silicone/polycarbonate. The membrane processes may be used for removing the nitrogen (N₂) from the methane or the natural gas. Some membranes are capable of separating more than one component, and therefore depending on the methane or natural gas composition, appropriate separation methods can be chosen.

The most important source of methane as used in stage (1) of the process is the natural gas. Thus, the process can comprise a preliminary stage prior to stage (1), comprising fractionating the components of the natural gas, so as to separate and to isolate (a) the methane from (b) the rest of the natural gas essentially comprising a mixture of the (C₂₊) alkanes, preferably the (C₂₋₆) alkanes. The methane thus isolated in the preliminary stage can be then used in stage (1).

The natural gas which can substitute the methane or from which the methane can be derived and used in stage (1) of the process, can be any natural gas, such that exploited directly on a deposit site. It is generally formed of a mixture of alkanes comprising mainly methane and, in weaker proportion, a mixture of the (C₂₊) alkanes, preferably the (C₂₋₆) alkanes, e.g. a mixture of ethane, propane, butanes, pentanes and hexanes, for example with the following molar percentages:
methane from 75 to 99%, preferably from 80 to 98%, particularly from 85 to 97%,
ethane from 1 to 15%, preferably from 1 to 10%, particularly from 1 to 7%,
propane from 0.1 to 10%, preferably from 0.1 to 5%, particularly from 0.1 to 3%,
butanes (n-butane and isobutene) from 0 to 3%, preferably from 0.01 to 2%, particularly from 0.01 to 1%,
pentanes (n-pentane and isopentane) from 0 to 2%, preferably from 0.01 to 1%, and
hexane(s) from 0 to 1%, preferably from 0.01 to 0.1%.

Sometimes, natural gas may also comprise traces of heptane(s) and/or higher alkanes.

Thus, the preliminary stage can comprise separating and isolating the methane from natural gas by fractionation. More particularly, the fractionation may comprise separating and isolating from the natural gas (a) the methane and preferably (b) the rest of said natural gas generally comprising a mixture of the (C₂₊) alkanes, e.g. a mixture of the C₂ to C₈, preferably C₂ to C₇, especially the C₂ to C₆ alklanes, i.e. the (C₂₋₆) alkanes, of the natural gas. The preliminary stage can be performed in various ways, either intermittently (or discontinuously), or preferably continuously. It can comprnse one or more methods suitable for fractionation of the natural gas, preferably chosen from any methods or combinations of methods for natural gas fractionation, in particular for fractionating the natural gas into (a) the methane and (b) the rest of said natural gas generally comprising a mixture of the (C₂₊) alkanes. The methods of fractionation can be preferably chosen from the following six types of fractionation:
fractionation by change of physical state, preferably of gaseous/liquid phase of the natural gas, in particular by distillation or by condensation (or liquefaction), more particularly by partial condensation (or partial liquefaction), preferably by means of distillation/condensation column or tower,
fractionation by molecular filtration, preferably by means of semi-permeable and selective membrane,
fractionation by adsorption, preferably by means of molecular sieve or any other adsorbent,
fractionation by absorption, preferably by means of absorbing oil,
fractionation by cryogenic expansion, preferably by means of expansion turbine, and
fractionation by compression, preferably by means of gas compressor.

The preliminary stage may comprise combining at least two successive fractionations of an identical or different type, preferably chosen from the six above-mentioned types of fractionation.

Any type of fractionation that is useful in general for separating methane from natural gas can be utilized in the preliminary stage.

In practice, the preliminary stage can be performed by change of the physical state, preferably of the gaseous/liquid phase of the natural gas, particularly by distillation or by condensation (or liquefaction), more particularly by partial condensation (or partial liquefaction), e.g. in at least one distillation/condensation column or tower, or in a distillation/condensation column reactor. The fractionation can be performed by refrigeration or cryogenic processes, preferably by cooling of the natural gas to a temperature at which the (C₂₊) alkanes condense, so as to condense said (C₂₊) alkanes, to separate and optionally to isolate them in liquid form from the methane which is thus separated and isolated preferably in gaseous form.

In practice, the preliminary stage may also be performed by molecular filtration, in particular by passing the natural gas through at least one semi-permeable and selective membrane, so as to arrest and optionally to isolate the (C₂₊) alkanes, and to allow the methane to pass through and thus to be separated and isolated preferably in gaseous form. The semi-permeable and selective membranes can be chosen from any membranes suitable for separating methane from natural gas, preferably chosen from zeolite membranes, porous alumina membranes, ceramic membranes, flowing liquid membranes and supported liquid membranes.

In practice, the preliminary stage may also be performed by adsorption, in particular by passing the natural gas onto at least one molecular sieve or any other adsorbent, so as to retain the methane (or alternatively the (C₂₊) alkanes) and to allow the rest of said natural gas comprising the (C₂₊) alkanes (or alternatively the methane) to pass through, and then by subjecting the methane (or alternatively the (C₂₊) alkanes) thus retained to at least one desorption step preferably performed by the TSA (“Temperature Swing Adsorption”) method or the PSA (“Pressure Swing Adsorption”) method, so as to separate and to isolate the methane and optionally the (C₂₊) alkanes. The adsorbents can be chosen from any adsorbents suitable for retaining methane (or alternatively the (C₂₊) alkanes), and preferably from molecular sieves, silica gels, activated charcoals and activated carbons.

In practice, the preliminary stage may also be performed by absorption, in particular by contacting the natural gas with an absorbing oil (e.g. in an absorption tower) so as to soak up the (C₂₊) alkanes and to form an oil/(C₂₊) alkane mixture, and then preferably by subjecting said oil/(C₂₊) alkane mixture to a temperature above the boiling point of the (C₂₊) alkanes, but below that of the oil (e.g. in a recovery tower), so as to separate and optionally to isolate the (C₂₊) alkanes from the oil and from the methane which is thus separated and isolated preferably in gaseous form.

In practice, the preliminary stage may also be performed by cryogenic expansion, in particular by dropping the temperature of the natural gas preferably to a temperature selected from −80 to −90° C., particularly around −85° C., e.g. by means of external refrigerants for pre-cooling the natural gas and then by means of an expansion turbine which rapidly expands the pre-cooled natural gas and causes the temperature to drop significantly, so as to condense the (C₂₊) alkanes, to separate and optionally to isolate them in liquid form from the methane which is thus separated and isolated preferably in gaseous form.

In practice, the preliminary stage may also be performed by compression, in particular by compressing the natural gas by means of a gas compressor, so as to condense the (C₂₊) alkanes, to separate and optionally to isolate them in liquid form from the methane which is thus separated and isolated preferably in gaseous form.

Stage (1) of the process comprises contacting methane with a metal catalyst (C1) capable of producing, in contact with alkane, reactions involving the splitting and recombining of carbon-carbon and/or carbon-hydrogen and/or carbon-metal bonds, so as to form by a non-oxidative methane coupling reaction a mixture (M1) comprising ethane and hydrogen.

The catalyst (C1) can be chosen from metal catalysts which are capable of alkane metathesis. It can be chosen particularly from supported metal clusters, and preferably from metal hydrides, organometallic compounds and organometallic hydrides, in particular supported on, and more specifically grafted onto, a solid support.

The catalyst (C1) can be chosen in particular from supported metal clusters comprising one or more metals of Groups 4, 5 and/or 6 of the Periodic Table of the Elements, preferably tantalum. The Periodic Table (here and hereafter) is that proposed by IUPAC in 1991 and, for example, published by CRC Press, Inc. (USA) in <<CRC Handbook of Chemistry and Physics>>, 76th Edition (1995-1996), by David P Lide. The supported metal clusters generally comprise a solid support and a metal cluster, and preferably comprise bonding between the metals of the metal cluster, and also bonding between the metal cluster and the solid support. Thus, the metal atom of the metal cluster can be bonded to at least one other metal atom of the metal cluster, preferably to six or fewer metal atoms of the metal cluster. The metal atom of the metal cluster can be bonded in addition to at least one hydrocarbon radical and/or to at least one hydrogen atom. The solid support can be chosen from a metal oxide, a refractory oxide, a molecular sieve, a zeolite, a metal phosphate and a material comprising a metal and oxygen, preferably silica. More particularly, the supported metal clusters can comprise tantalum (as a metal cluster) and silica (as a solid support), and preferably comprise bonding between tantalum and tantalum, and also bonding between tantalum and silica, in particular bonding between tantalum and oxygen on the silica surface. The catalyst (C1) can be chosen from the supported metal clusters described in International Patent Application WO 2006/060692, and prepared according to any methods described in said Application.

The catalyst (C1) is preferably chosen from metal hydrides, organometallic compounds and organometallic hydrides, particularly supported on, and more specifically grafted onto, a solid support. It can comprise at least one metal chosen from lanthanides, actinides and metals of Groups 2 to 12, preferably transition metals of Groups 3 to 12, more particularly Groups 3 to 10 of the Periodic Table of the Elements. In particular, the metal of the catalyst can be chosen from scandium, yttrium, lanthanum, titanium, zirconium, hafnium, vanadium, niobium, tantalum, chromium, molybdenum, tungsten, rhenium, ruthenium, osmium, nickel, iridium, palladium, platinum, cerium and neodymium. It can be chosen, preferably, from yttrium, titanium, zirconium, hafnium, vanadium, niobium, tantalum, chromium, molybdenum, tungsten, rhenium, ruthenium and platinum, and more especially from niobium, tantalum, molybdenum, tungsten and rhenium.

The catalyst (C1) chosen from metal hydrides and preferably from organometallic compounds and organometallic hydrides, can comprise in particular at least one hydrocarbon radical (R), either saturated or unsaturated, linear or branched, having preferably from 1 to 20, in particular from 1 to 14 carbon atoms. The hydrocarbon radicals (R) can be chosen from alkyl radicals, in particular either saturated or unsaturated, linear or branched, aliphatic or alicyclic, for examples alkyl, alkylidene or alkylidyne radicals, particularly from C₁ to C₁₀, from aryl radicals, in particular from C₆ to C₁₂, and from aralkyl, aralkylidene or aralkylidyne radicals, particularly from C₇ to C₁₄. Thus, the metal atom of the catalyst (C1) can preferably be bonded to at least one hydrogen atom and/or to at least one hydrocarbon radical (R), in particular by a single, double or triple carbon-metal bond.

The catalyst (C1) preferably selected from metal hydrides, organometallic compounds and organometallic hydrides, can furthermore comprise one or more ligands, such as “ancillary” ligands, preferably comprising at least one oxygen atom and/or at least one nitrogen atom, and particularly chosen from oxo, alkoxo, aryloxo, aralkyloxo, nitrido, imido and amido ligands. Generally, by oxo, alkoxo, aryloxo, aralkyloxo, nitrido, imido and amido ligands are respectively meant:

a divalent oxo radical of general formula: ═O

a monovalent alkoxo, aryloxo or aralkyloxo radical of general formula: —OR′

a trivalent nitrido radical of general formula: ≡N

a divalent imido radical of general formula: ═NR″, and

a monovalent amido-radical of general formula: —NR¹R²,

general formulae in which Q represents an oxygen atom, N represents a nitrogen atom, R′ represents an hydrogen atom or a monovalent hydrocarbon radical selected respectively from alkyl, aryl and aralkyl radicals, R″ represents an hydrogen atom or a monovalent hydrocarbon radical, and R¹ and R², being identical or different, represent an hydrogen atom or a monovalent hydrocarbon radical. More particularly, R′, R″, R¹ and R² can be a monovalent hydrocarbon radical, either saturated or unsaturated, linear or branched, comprising from 1 to 20, preferably from 1 to 14 carbon atoms, and particularly chosen from alkyl radicals, preferably from C₁ to C₁₀, aralkyl radicals preferably from C₇ to C₁₄, and aryl radicals, preferably from C₆ to C₁₂.

Thus, when the catalyst (C1) selected in particular from metal hydrides, organometallic compounds and organometallic hydrides comprises at least one of the above-mentioned ligands, the metal of the catalyst is not only bonded to at least one hydrogen atom and/or to at least one hydrocarbon radical (R), but also to at least one of these ligands, e.g. to the O atom of the oxo radical by a double bond, or to the O atom of the alkoxo, aryloxo or aralkyloxo radical (OR′) by a single bond, or to the N atom of the nitrido radical by a triple bond, or to the N atom of the imido radical (NR″) by a double bond, or to the N atom of the amido radical (NR¹R²) by a simple bond. Generally the presence of the oxo, alkoxo, aryloxo, aralkyloxo, nitrido, imido and/or amido ligands in the catalyst (C1) favourably influences the behaviour of the catalyst in the reactions of stage (1) particularly producing ethane and hydrogen.

In addition, the catalyst (C1) chosen from metal hydrides, organometallic compounds and organometallic hydrides is preferably supported on, or more particularly grafted onto, a solid support that can be chosen from metal or refractory oxides, sulphides, carbides, nitrides and salts, and from carbon, mesoporous materials, organic/inorganic hybrid materials, metals and molecular sieves. The solid support is in particular chosen from silicas, aluminas, silica-aluminas and aluminium silicates, either simple or modified by other metals, and from zeolites, clays, titanium oxide, cerium oxide, magnesium oxide, niobium oxide, tantalum oxide and zirconium oxide. It can be chosen, preferably, from silicas, aluminas, silica-aluminas and aluminium silicates, either simple or modified by other metals. The solid support can have a specific surface area (B.E.T.) measured according to the standard ISO 9277 (1995) which is chosen in a range of from 0.1 to 5000 m²/g, preferably from 1 to 3000 m²/g, more particularly from 1 to 1000 m²/g. It is preferred to use a catalyst (C1) grafted onto a solid support and in which the metal atom can be bonded to at least one atom of hydrogen, to at least one hydrocarbon radical (R), and also to at least one of the atoms of the support, in particular to at least one atom of oxygen, sulphur, carbon or nitrogen of the support, in particular when the support is chosen from metal or refractory oxides, sulphides, carbides, nitrides and salts. The catalyst (C1) can be preferably a metal hydride or an organometallic hydride grafted onto a solid support, more particularly chosen from niobium hydrides or organometallic niobium hydrides grafted onto silica, tantalum hydrides or organometallic tantalum hydrides grafted onto silica, molybdenum hydrides or organometallic molybdenum hydrides grafted onto silica, tungsten hydrides or organometallic tungsten hydrides grafted onto alumina, and rhenium hydrides or organometallic rhenium hydrides grafted onto silica.

The catalyst (C1) selected from metal hydrides, organometallic compounds and organometallic hydrides can be in particular chosen from the metal catalysts described in the International Patent Applications WO 98/02244, WO 03/061823 and WO 2004/089541, and prepared according to any of the methods described in said Applications.

In stage (1), the methane can be used as the only or substantially the only starting alkane, e.g. in the form of a mixture of methane with less than 1% by mole of one or more other alkanes preferably selected from the (C₂₊), particularly the. (C₂₋₆) alkanes as present in the natural gas. In this case, the methane used alone or substantially alone as the starting material in stage (1) can be derived from the natural gas and obtained preferably by fractionating the natural gas, and separating and isolating the methane, particularly by submitting the natural gas to the above-mentioned preliminary stage.

Thus, in stage (1), when the methane is used as the only or substantially the only starting alkane, the methane in contact with the catalyst (C1) can react essentially with itself, and generally produces ethane and hydrogen by a non-oxidative methane coupling reaction, in particular described according to the equation (1):

2CH₄→C₂H₆+H₂ (1)

In stage (1), the methane can also be used in the form of a mixture of methane, preferably in a major molar proportion, with one or more other starting alkanes in particular selected from the other alkanes present in the natural gas, particularly the (C₂₊), preferably the (C₂₋₆) alkanes of the natural gas, especially in molar proportions similar or identical to those existing in the natural gas. Thus, the mixture of methane with one or more other starting alkanes may comprise from more than 50% to 99% by mole of methane, preferably from 60% to 99% by mole of methane, particularly from 75% to 99% by mole of methane. More particularly, the methane can be used in stage (1) in the form of the natural gas, and thus the methane can be substituted by the natural gas as the starting alkane material in stage (1). Without wishing to be bound by any theory relating to reaction mechanisms, it is believed that; when the methane is used with one or more other starting alkanes or preferably is substituted by the natural gas in stage (1), a plurality of simultaneous reactions can be involved and lead finally to a mixture (M1) comprising ethane and hydrogen.

Thus, stage (1) can involve in the presence of the catalyst (C1) simultaneously:
(i) the non-oxidative methane coupling reaction, such as described by equation (1), and
(ii) (ii) methane-olysis reactions between the methane and the other starting alkanes present in the methane mixture or in the natural gas, leading particularly to the production of ethane.

More particularly, the methane-olysis reactions can occur in stage (1) between the methane and each of the other starting alkanes, such as the (C₂₊) alkanes of the natural gas, preferably the (C₃₊) alkanes (having 3 carbon atoms and more) of the natural gas, especially the (C₃₋₆) alkanes, e.g. propane, butanes, pentanes and hexanes, for instance according to the following methane-olysis reactions:

methane-olysis reaction of propane according to equation (2):

CH₄+C₃H₈→2C₂H₆ (2)

methane-olysis reactions of butanes according to equations (3), (2) and (4):

CH₄+C₄H₁₀→C₂H₆+C₃H₈ (3)

CH₄+C₃H₈→2C₂H₆ (2)

that is to say overall: 2CH₄+C₄H₁₀→3C₂H₆ (3)+(2)=(4)

methane-olysis reactions of pentanes according to equations (5), (4) and (6):

CH₄+C₅H₁₂→C₂H₆+C₄H₁₀ (5)

2CH₄+C₄H₁₀→3C₂H₆ (4)

that is to say overall: 3CH₄+C₅H₁₂→4C₂H₆ (5) +(4) =(6)

and methane-olysis reactions of hexanes according to equations (7), (6) and (8):

CH₄+C₆H₁₄→C₂H₆+C₅H₁₂ (7)

3CH₄+C₅H₁₂→4C₂H₆ (6)

that is to say overall: 4CH₄+C₆H₁₄→5C₂H₆ (7)+(6)=(8)

Thus, the methane-olysis reactions of propane, butanes, pentanes and hexanes can be summarized overall according to equation (9) which is the sum of equations (2), (4), (6) and (8), as follows:

10CH₄+C₃H₈+C₄H₁₀+C₅H₁₂+C₆H₁₄→14C₂H₆ (2)+(4)+(6)+(8)=(9)

Stage (1) thus leads to formation of a mixture (M1) comprising ethane and hydrogen, and optionally unreacted methane. When in stage(1) the methane is used as a mixture of the methane with one or more other starting alkanes, or in the form of the natural gas, the mixture (M1) may in addition comprise one or more unreacted starting alkanes, in particular the (C₃₊), preferably the (C₃₋₆) alkanes, such as those present in the natural gas (e.g. propane, butanes, pentanes and hexanes), existing particularly at low concentrations. Under the operating conditions of stage (1), the mixture (M1) is generally obtained in gaseous form or in mixed gaseous/liquid form.

Stage (1) can be performed under conditions specially suited to promoting the development of the non-oxidative methane coupling reaction and optionally the methane-olysis reactions towards an optimum production of ethane. Thus, it can be performed at a temperature chosen in a range of from 50 to 700° C., preferably 150 to 700° C., particularly 70 to 650° C., more particularly 200 to 650° C., in particular 100 to 600° C., especially 300 to 600° C. It can also be performed under an absolute total pressure chosen in a range of from 0.1 to 100 MPa, preferably 0.1 to 50 MPa, in particular 0.1 to 30 MPa, more particularly 0.1 to 20 MPa. It can also be performed in the presence of at least one inert agent, either liquid or gaseous, in particular of an inert gas chosen from nitrogen, helium and argon. Stage (1) can also be performed advantageously under a partial pressure of methane chosen in a range of from 0.1 to 50 MPa, preferably 0.1 to 30 MPa, particularly 0.2 to 20 MPa, especially 0.5 to 10 MPa, in particular when in stage (1) the methane is used as a mixture of the methane with one or more other starting alkanes, or is substituted by the natural gas.

Stage (1) can be performed in various ways in a reaction zone (Z1), either intermittently (or discontinuously), or preferably continuously.

In the present description, when the methane is hereafter mentioned to be used in stage (1), it is understood that the methane can be substituted by the above-mentioned mixture of the methane with one or more other starting alkanes, or else by the natural gas.

Stage (1) can be performed, for example, by adding the methane to the catalyst (C1), or by adding the catalyst (C1) to the methane, or else by adding the methane and the catalyst (C1) simultaneously into a reaction zone (Z1).

In addition, the reaction zone (Z1) can comprise at least one reactor chosen from static reactors, recycling reactors and dynamic reactors. For example, stage (1) can be performed in a static reactor containing fixed quantities of the catalyst (C1) and of the methane. Stage (1) can also be performed in a recycling reactor into which at least one component of the mixture (M1), for example ethane or optionally unreacted methane, or of the mixture of the methane with one or more other starting alkanes, or else of the natural gas, can be recycled, preferably continuously, after it has been preferably separated from the mixture (M1) beforehand. Stage (1) can also be performed in a dynamic reactor containing the catalyst (C1), in the form of solid particles, or as a bed or a distillation packing, through which the methane can pass or circulate, preferably continuously.

In practice, stage (1) can be performed in a reaction zone (Z1) comprising at least one reactor chosen from tubular (or multi-tubular) reactors, distillation/condensation column reactors, slurry reactors, fluidised bed reactors, mechanically agitated bed reactors, fluidised and mechanically agitated bed reactors, fixed bed reactors, moving bed reactors and circulating bed reactors. The catalyst (C1) is generally solid, and can optionally be mixed with an inert solid agent chosen in particular from silicas, aluminas, silica-aluminas and aluminium silicates, either simple or modified by other metals. The catalyst (C1) can be arranged inside the tubes of a tubular (or multi-tubular) reactor. It can also be arranged inside a distillation/condensation column reactor, wherein it is preferably a component of a distillation/condensation system functioning as both a catalyst and a distillation/condensation packing, i.e. a packing for a distillation/condensation column having both a distillation/condensation function (as in stage (2)) and a catalytic function (as in stage (1)): for example, rings, saddles, balls, granulates, irregular shapes, granulates, sheets, tubes, spirals, packed in bags, as described in American Patent U.S. Pat. No. 4,242,530. The catalyst (C1) can also form the bed of a fluidised and/or mechanically agitated bed, fixed bed, moving bed or circulating bed of said reactors. The methane can be introduced in the form of a stream, preferably continuously, into one of said reactors, and in particular pass or circulate into the tubes or through the bed or the distillation packing of said reactors. In order to promote the development of the methane coupling reaction and optionally the methane-olysis reactions towards an optimum production of the ethane, stage (1) can be advantageously performed by withdrawing at least one component of the mixture (M1) such as the ethane and/or the unreacted methane out of the reaction zone (Z1), preferably continuously.

Thus, for example, stage (1) can be performed:

(i) by continuously introducing the methane into the reaction zone (Z1) containing the catalyst (C1), so as to form continuously the mixture (M1) comprising ethane, hydrogen, optionally the unreacted methane and optionally at least one unreacted component of the mixture of the methane with one or more other starting alkanes, or optionally at least one unreacted component of the natural gas, and

(ii) by continuously withdrawing at least a part of the mixture (M1) out of the reaction zone (Z1), so as to submit said part of the mixture (M1) optionally to stage (2) for separating the ethane from the mixture (M1) and isolating the ethane from the rest of the mixture, or directly to stage (3), and preferably to return the rest of the mixture, preferably the unreacted methane, and optionally the other non-separated part of the mixture (M1) into the reaction zone (Z1), particularly in order to pursue the non-oxidative methane coupling reaction and optionally the methane-olysis reactions in stage (1).

The process then optionally or preferably can comprise a stage (2) comprising fractionating the mixture (M1) so as to separate and to isolate the ethane. When the methane is used in stage (1) in the form of the above-mentioned mixture of the methane with one or more other starting alkanes or the natural gas, stage (2) preferably can also comprise fractionating the mixture (M1) so as to separate the ethane and one or more (C₃₊), preferably (C₃₋₆) alkanes, e.g. propane, butanes, pentanes and hexanes, and to isolate the ethane and said (C₃₊), preferably (C₃₋₆) alkanes, either separately or in the form of a mixture, which can preferably be used subsequently in stage (3).

Stage (2) can be performed in various ways, either intermittently (or discontinuously), or preferably continuously. It can comprise one or more methods of fractionation of the mixture (M1), in particular chosen from any known methods or combinations of methods for fractionation of such a mixture, preferably from the following six types of fractionation:
fractionation by change of physical state, preferably of liquid/gaseous phase of the mixture, in particular by distillation or by condensation, preferably by partial condensation, preferably by means of distillation/condensation column or tower,
fractionation by molecular filtration, preferably by means of semi-permeable and selective membrane,
fractionation by adsorption, preferably by means of molecular sieve or any other adsorbent,
fractionation by absorption, preferably by means of absorbing oil,
fractionation by cryogenic expansion, preferably by means of expansion turbine, and
fractionation by compression, preferably by means of gas compressor.

Any type of fractionation that is useful in general for separating ethane optionally in admixture with one or more (C₃₊), preferably (C₃₋₆) alkanes, from hydrogen and optionally methane, can be utilized in stage (2).

Stage (2) thus makes it possible firstly to separate the ethane optionally in admixture with one or more (C₃₊), preferably (C₃₋₆) alkanes from the mixture (M1), and to isolate the ethane optionally in said admixture. Furthermore, it can also permit-the hydrogen to be separated and isolated from the mixture (M1) and preferably to be subsequently used for any applications or stages directly used or not in the present process, e.g. for producing energy. It can also permit the unreacted methane to be separated and isolated from the mixture (M1) and preferably to be recycled into stage (1).

Stage (2) can be performed in a fractionation zone (F1) which is either separate and distinct from the reaction zone (Z1) wherein stage (1) is performed, or arranged in a part of said reaction zone (Z1), preferably in a distillation/condensation column reactor as previously mentioned and described in American Patent U.S. Pat. No. 4,242,530.

Thus, a distillation/condensation column reactor can be advantageously used both for stage (1) and stage (2), and contain the catalyst (C1). More particularly, the catalyst (C1) can be arranged inside the distillation/condensation column reactor, as a component of a distillation/condensation system functioning as both a catalyst and a distillation/condensation packing. Thus, stages (1) and (2) can be simultaneously performed in at least one distillation/condensation column reactor comprising both a reaction zone and a fractionation zone. In the reaction zone, the contacting of the methane with the catalyst (C1) is performed as in stage (1), so as to form the mixture (M1) preferably in a liquid/gaseous phase. Simultaneously, in the fractionation zone, the separation of ethane optionally in admixture with one or more (C₃₊), preferably (C₃₋₆) alkanes from the mixture (M1) is performed as in stage (2), preferably by distillation or by condensation, so as to isolate the ethane optionally in said admixture.

More generally, a fractionation of the mixture (M1) can be performed by change of the physical state, preferably of the liquid/gaseous phase of the mixture, particularly by distillation or by condensation, in particular by partial condensation, e.g. in at least one distillation/condensation column or tower, or in a distillation/condensation column reactor. The fractionation can be performed in particular by refrigeration or cryogenic processes, preferably by at least one cooling of the gaseous mixture (M1) to at least one temperature at which at least one component of the mixture condenses and the rest of the mixture remains in gaseous form, so as to separate and to isolate said component(s) from said rest of the mixture.

Generally, a fractionation of the mixture (M1) can also be performed by molecular filtration, preferably by passing the gaseous mixture (M1) through at least one semi-permeable and selective membrane, so as to arrest and to isolate at least one component of the mixture and to allow the rest of the mixture to pass through.

Generally, the fractionation of the mixture (M1) can also be performed by adsorption, preferably by passing the gaseous mixture (M1) over at least one molecular sieve or any other adsorbent, so as to retain at least one component of the mixture and to allow the rest of the mixture to pass through, the component(s) thus retained being then submitted to at least one desorption step, preferably by the TSA method (“Temperature Swing Adsorption”) or the PSA method (“Pressure Swing Adsorption”), so as to isolate said component(s).

Generally, a fractionation of the mixture (M1),can also be performed by absorption, in particular by contacting the gaseous mixture (M1) with an absorbing oil, e.g. in an absorption tower, so as to soak up at least one component of the mixture, to form an oil/component mixture and to allow the rest of the gaseous mixture to be free and not absorbed, then by subjecting said oil/component mixture,.e.g. in a recovery tower, to a temperature above the boiling point of the component, but below that of the oil, so as to separate and to isolate said component from the oil.

Generally, a fractionation of the mixture (M1) can also be performed by cryogenic expansion, in particular by dropping the temperature of the gaseous mixture (M1) to a temperature so as to condense at least one component of the mixture while maintaining the rest of the mixture in gaseous form, e.g. by means of an expansion turbine which rapidly expands the gaseous mixture and causes a significant drop of the temperature.

Generally, a fractionation of the mixture (M1) can also be performed by compression, in particular by compressing the gaseous mixture (M1) e.g. by means of a gas compressor, so as to condense at least one component of the mixture, to separate and to isolate said component, and to allow the rest of the mixture to be maintained in gaseous form.

For example, a fractionation of the gaseous mixture (M1) can be performed by change of the gaseous phase of the mixture, e.g. in at least one distillation/condensation column or tower, or in a distillation/condensation column reactor, preferably by at least one cooling of the gaseous mixture to a temperature at which ethane and optionally one or more (C₃₊), preferably (C₃₋₆) alkanes condense, so as to condense the ethane optionally in admixture with said (C₃₊), preferably (C₃₋₆) alkanes, to separate and to isolate it (or them) in liquid form from the rest of the gaseous mixture comprising the hydrogen and optionally the unreacted methane.

For example, a fractionation of the gaseous mixture (M1) can also be performed by molecular filtration of the mixture, preferably by passing the gaseous mixture through at least one semi-permeable and selective membrane, so as to arrest and to isolate the ethane optionally in admixture with one or more (C₃₊), preferably (C₃₋₆) alkanes, and to allow the rest of the gaseous mixture comprising the hydrogen and optionally the unreacted methane, to pass through. The semi-permeable and selective membranes can be chosen from any those that are suitable for arresting and separating ethane optionally in admixture with one or more (C₃₊), preferably (C₃₋₆) alkanes from hydrogen and optionally methane. They can be preferably chosen from zeolite membranes, porous alumina membranes, ceramic membranes, flowing liquid membranes and supported liquid membranes.

For example, a fractionation of the gaseous mixture (M1) can also be performed by adsorption, preferably by passing the gaseous mixture over at least one molecular sieve or any other adsorbent, so as to retain the ethane optionally in admixture with one or more (C₃₊), preferably (C₃₋₆) alkanes, and to allow the rest of the gaseous mixture comprising the hydrogen and optionally the unreacted methane to pass through, and then to subject the ethane (optionally in said admixture) thus retained to a desorption step, for instance performed by the TSA or the PSA method, so as to isolate the ethane optionally in said admixture. The adsorbents can be chosen from any those that are capable of retaining and separating ethane optionally in admixture with one or more (C₃₊), preferably (C₃₋₆) alkanes from hydrogen and optionally from methane. They can be preferably chosen from molecular sieves, silica gels, activated charcoals and activated carbons.

For example, a fractionation of the gaseous mixture (M1) can also be performed by absorption, preferably by contacting the gaseous mixture (M1) with an absorption oil, e.g. in an absorption tower, so as to soak up the ethane (optionally in admixture with one or more (C₃₊), preferably (C₃₋₆) alkanes) from the mixture (M1) and to form an oil/ethane mixture, and then preferably by subjecting said mixture, e.g. in a recovery tower, to a temperature above the boiling point of the ethane and optionally of the (C₃₊), preferably the (C₃₋₆) alkanes, but below that of the oil, so as to separate and to isolate the ethane optionally in said admixture from the oil and from the rest of the gaseous mixture comprising the hydrogen and optionally the unreacted methane.

For example, a fractionation of the gaseous mixture (M1) can also be performed by cryogenic expansion, preferably by dropping the temperature of the gaseous mixture (M1) in particular by use of external refrigerants for pre-cooling the gaseous mixture and then by means of an expansion turbine which rapidly expands the pre-cooled gaseous mixture and causes the temperature to drop significantly, so as to condense the ethane and optionally one or more (C₃₊), preferably (C₃₋₆) alkanes, to separate and to isolate it (or them) from the rest of the gaseous mixture comprising the hydrogen and optionally the unreacted methane.

For example, a fractionation of the gaseous mixture (M1) can also be performed by compression, preferably by compressing the gaseous mixture (M1), e.g. by means of a gas compressor, so as to condense the ethane and optionally one or more (C₃₊), preferably (C₃₋₆) alkanes, to separate and to isolate it (or them) in liquid form from the rest of the gaseous mixture comprising the hydrogen and optionally the unreacted methane.

Stage (2) can be performed advantageously by a double. (or multiple) fractionation of the mixture (M1) comprising a combination of two (or more) successive fractionations of an identical or different type, chosen from the six above-mentioned types of fractionation, so as in particular to separate and to isolate (a) ethane optionally in admixture with one or more (C₃₊), preferably (C₃₋₆) alkanes, (b) hydrogen and (c) unreacted methane.

The unreacted methane can be advantageously separated and isolated in stage (2) and then recycled into stage (1), particularly for pursuing the non-oxidative methane coupling reaction and optionally the methane-olysis reactions. Moreover, it may also be particularly advantageous to separate and to isolate hydrogen from the mixture (M1), and then to use it in one or more applications or stages, distinct or not from the present process. For example, the hydrogen thus isolated can be used as an agent for activation or regeneration of the catalyst(s) (C1) and/or (C2) used in the process. The activation or the regeneration of the catalyst can be carried out by contacting the catalyst with the hydrogen, during one of the stages of the process, preferably during stage (3) for the simultaneous ethane self- and cross-metathesis reactions, or in a stage distinct and separate from the process. The hydrogen can also be used highly advantageously in other applications or stages, distinct or not from the present process, for example as a fuel for producing thermal and/or electrical energies, in particular as a fuel in hydrogen fuel cells for producing IS electrical energy, such energies being preferably employed for running the present process, or as a fuel for automobile, or as a reagent in a chemical, petrochemical or refinery plant. It is remarkable to notice that the hydrogen produced and isolated by the present process can generally supply all the energy needed to run said process.

For example, it is possible to perform a double fractionation by subjecting the gaseous mixture (M1) comprising ethane optionally in admixture with one or more (C₃₊), preferably (C₃₋₆) alkanes, hydrogen and unreacted methane, to a combination of two successive fractionations of one and the same type, namely by change of physical state, preferably of the liquid/gaseous phase of the mixture, e.g. in one or more distillation/condensation columns or towers, or in a distillation/condensation column reactor, for example as follows:

(i) by a first cooling of the gaseous mixture (M1) to a temperature at which the ethane and optionally one or more (C₃₊), preferably (C₃₋₆) alkanes condense, so as to condense the ethane optionally with said (C₃₊), preferably (C₃₋₆) alkanes, to separate and to isolate it (or them) in liquid form from the rest of the gaseous mixture comprising the hydrogen and the unreacted methane, and

(ii) by a second cooling of said rest of the gaseous mixture to a temperature at which the methane condenses, so as to condense the unreacted methane, to separate and to isolate it in liquid form from the hydrogen which is preferably isolated in its turn in, gaseous form.

It is also possible to perform a double fractionation by subjecting the above-mentioned gaseous mixture (M1) to a combination of two successive fractionations of one and the same type, namely by molecular filtration:

(i) by passing the gaseous mixture (M1) through a first semi-permeable and selective membrane, so as to arrest and to isolate the ethane optionally in admixture with one or more (C₃₊), preferably (C₃₋₆) alkanes, and to allow the rest of the gaseous mixture comprising the hydrogen and the unreacted methane to pass through, and

(ii) by passing said rest of the gaseous mixture through a second semi-permeable and selective membrane, so as to arrest and to isolate the methane, and to allow the hydrogen to pass through and preferably to be isolated in its turn in gaseous form.

It is also possible to perform a double fractionation by subjecting the above-mentioned gaseous mixture (M1) to a combination of two successive fractionations of one and the same type, namely by adsorption:

(i) by passing the gaseous mixture (M1) onto a first molecular sieve or any other adsorbent, so as to retain the ethane optionally in admixture with one or more (C₃₊), preferably (C₃₋₆) alkanes, and to allow the rest of the gaseous mixture comprising the hydrogen and the unreacted methane to pass through, then

(ii) by passing said rest of the gaseous mixture onto a second molecular sieve or any other adsorbent, so as to retain the unreacted methane (or the hydrogen), and to allow the hydrogen (or the unreacted methane) to pass through, and

(iii) by respectively submitting the ethane optionally in said admixture and the methane (or the hydrogen) thus retained to desorption steps, preferably performed by the TSA or the PSA method, so as respectively to isolate the ethane optionally in said admixture and the unreacted methane (or the hydrogen).

For example, it is also possible to perform a double fractionation by subjecting the above-mentioned gaseous mixture (M1) to a combination of two successive fractionations of one and the same type, namely by absorption, e.g. in one or more absorption towers:
(i) by contacting the gaseous mixture (M1) with a first absorbing oil, so as to soak up the ethane (optionally in admixture with one or more (C₃₊), preferably (C₃₋₆) alkanes) from the mixture, to form an oil/ethane mixture, and to allow the rest of the gaseous mixture comprising the unreacted methane and the hydrogen to be free in gaseous form,
(ii) by contacting said rest of the gaseous mixture with a second absorbing oil, so as to soak up the methane, to form an oil/methane mixture, and to allow the hydrogen to be free in gaseous form and preferably to be isolated, and
(iii) by subjecting said oil/ethane mixture to a temperature above the boiling point of the ethane and optionally of the (C₃₊), preferably (C₃₋₆) alkanes, but below that of the oil, e.g. in a recovery tower, so as to separate and to isolate the ethane optionally in said admixture from the oil, and preferably by subjecting said oil/methane mixture to a temperature above the boiling point of the methane, but below that of the oil, e.g. in a recovery tower, so as to separate and to isolate the unreacted methane from the oil.

For example, it is also possible to perform a double fractionation by subjecting the above-mentioned gaseous mixture (M1) to a combination of two successive fractionations of one and the same type, namely by cryogenic expansion:
(i) by dropping the temperature of the gaseous mixture (M1) by means of an expansion turbine, so as to condense the ethane and optionally one or more (C₃₊), preferably (C₃₋₆) alkanes, to separate and to isolate it (or them) in liquid form from the rest of the gaseous mixture comprising the hydrogen and the unreacted methane, and
(ii) by dropping the temperature of said rest of the gaseous mixture by means of an expansion turbine, so as to condense the methane, to separate and to isolate it in liquid form from the hydrogen which is preferably isolated in its turn in gaseous form.

For example, it is also possible to perform a double fractionation by subjecting the above-mentioned gaseous mixture (M1) to a combination of two successive fractionations of one and the same type, namely by compression:
(i) by compressing the gaseous mixture (M1), e.g. by means of a gas compressor, so as to condense the ethane and optionally one or more (C₃₊), preferably (C₃₋₆) alkanes, to separate and to isolate it (or them) in liquid form from the rest of the gaseous mixture comprising the unreacted methane and the hydrogen, and
(ii) by compressing said rest of the gaseous mixture, e.g. by means of a gas compressor, so as to condense the methane, to separate and to isolate it in liquid form from the hydrogen which is preferably isolated in its turn in gaseous form.

It is also possible to perform a double (or multiple) fractionation by subjecting the gaseous mixture (M1) comprising the ethane optionally in admixture with one or more (C₃₊), preferably (C₃₋₆) alkanes, the hydrogen and the unreacted methane, to a combination of two (or more) successive fractionations of a different type, chosen from the six above-mentioned types of fractionation.

A double fractioning of the above-mentioned gaseous mixture (M1) can, for example, be performed by combining two successive fractionations of a different type, namely:

the one by change of physical state, preferably of liquid/gaseous phase of the mixture, such as by distillation or by condensation, and the other by molecular filtration, preferably by means of a semi-permeable and selective membrane, or

the one by change of physical state, preferably of liquid/gaseous phase of the mixture, such as by distillation or by condensation, and the other by adsorption, preferably by means of a molecular sieve or any other adsorbent, or

the one by change of physical state, preferably of liquid/gaseous phase of the mixture, such as by distillation or by condensation, and the other by absorption, preferably by means of an absorbing oil, or

the one by change of physical state, preferably of liquid/gaseous phase of the mixture, such as by distillation or by condensation, and the other by cryogenic expansion, preferably by means of an expansion turbine, or

the one by change of physical state, preferably of liquid/gaseous phase of the mixture, such as by distillation or by condensation, and the other by compression, preferably by means of a gas compressor, or

the one by molecular filtration, preferably by means of a semi-permeable and selective membrane, and the other by adsorption, preferably by means of a molecular sieve or any other adsorbent, or

the one by molecular filtration, preferably by means of a semi-permeable and selective membrane, and the other by absorption, preferably by means of an absorbing oil, or

the one by molecular filtration, preferably by means of a semi-permeable and selective membrane, and the other by cryogenic expansion, preferably by means of an expansion turbine, or

the one by molecular filtration, preferably by means of a semi-permeable and selective membrane, and the other by compression, preferably by means of a gas compressor, or

the one by adsorption, preferably by means of molecular sieve or any other adsorbent, and the other by absorption, preferably by means of an absorbing oil, or

the one by adsorption, preferably by means of molecular sieve or any other adsorbent, and the other by cryogenic expansion, preferably by means of an expansion turbine, or

the one by adsorption, preferably by means of molecular sieve or any other adsorbent, and the other by compression, preferably by means of a gas compressor, or

the one by absorption, preferably by means of an absorbing oil, and the other by cryogenic expansion, preferably by means of an expansion turbine, or

the one by absorption, preferably by means of an absorbing oil, and the other by compression, preferably by means of a gas compressor, or

the one by cryogenic expansion, preferably by means of an expansion turbine, and the other by compression, preferably by means of a gas compressor.

Each of said combinations can be performed in a chronological order or in a reverse order.

Thus, for example, the two successive fractionations of a different type can be performed advantageously:

(i) by cooling the gaseous mixture (M1) to a temperature at which the ethane and optionally one or more (C₃₊), preferably (C₃₋₆) alkanes condense, so as to condense the ethane in admixture with said (C₃₊), preferably (C₃₋₆) alkanes, to separate and to isolate it (or them) in liquid form from the rest of the gaseous mixture comprising the hydrogen and the unreacted methane, and

(ii) by passing said rest of the gaseous mixture through a semi-permeable and selective membrane, so as to arrest and to isolate the unreacted methane, and to allow the hydrogen to pass through and preferably to be isolated in its turn in gaseous form.

It is also possible, for example, to perform the two above-mentioned successive fractionations in a reverse order, that is to say:

(i) by passing the gaseous mixture (M1) through a semi-permeable and selective membrane, so as to arrest and to isolate the ethane optionally in admixture with one or more (C₃₊), preferably (C₃₋₆) alkanes, and to allow the rest of the gaseous mixture comprising the hydrogen and the unreacted methane to pass through, and

(ii) by cooling said rest of the gaseous mixture to a temperature at which the methane condenses, so as to condense the unreacted methane, to separate and to isolate it in liquid form from the hydrogen which is preferably isolated in its turn in gaseous form.

Thus, e.g. by means of a double (or multiple) fractionation such as one of those described above, the methane thus isolated in stage.(2) is preferably recycled into stage (1), particularly for pursuing the non-oxidative methane coupling reaction and optionally the methane-olysis reactions. Furthermore, the hydrogen thus isolated in stage (2) can be used advantageously in one of the above-mentioned applications, such as an agent of activation or regeneration of the catalyst(s) (C1) and/or (C2), or as a fuel for producing thermal and/or electrical energies, in particular as a fuel in hydrogen fuel cells for producing electrical energy, such energies being preferably employed to run the present process, or as a fuel for automobile, or as a reagent in a chemical, petrochemical or refinery plant.

The process then comprises a stage (3) for producing and isolating the higher alkane mixture comprising the (C₃₊), preferably the (C₄₊), especially the (C₅₊) alkanes, preferably in liquid form. More particularly, stage (3) comprises, preferably simultaneously:

(i) contacting the mixture (M1) obtained in stage (1) or preferably the ethane isolated in stage (2) with a metal catalyst (C2) capable of producing, in contact with alkane, reactions involving the splitting and re-combining of carbon-carbon and/or carbon-hydrogen and/or carbon-metal bonds, so as to form by simultaneous ethane self- and cross-metathesis reactions a mixture (M2) comprising methane and the higher alkane mixture comprising the (C₃₊), preferably (C₄₊), especially (C₅₊) alkanes, and

(ii) fractionating the mixture (M2) so as to separate and to isolate the higher alkane mixture comprising the (C₃₊), preferably the (C₄₊), especially the (C₅₊) alkanes, preferably in liquid form, and particularly to separate and to isolate the methane which is recycled into stage (1).

The catalyst (C2) can be identical to or preferably different from the catalyst (C1) used in stage (1), and can conform in particular to the general definition of metal catalysts given above for the catalyst (C1). In the case when the catalyst (C2) is different from the catalyst (C1), it might be possible to use directly in stage (3) the mixture (M1) obtained in stage (1), instead of using the ethane isolated in stage (2).

In particular, the catalyst (C2) can be chosen from metal catalysts that are capable of alkane metathesis. It can be chosen from supported metal clusters, and preferably from metal hydrides, organometallic compounds and organometallic hydrides, particularly supported, on, or more specifically grafted onto, a solid support.

The catalyst (C2) can be chosen from supported metal clusters comprising one or more metals of Groups 4, 5 and/or 6 of the Periodic Table of the Elements, preferably tantalum. The supported metal clusters generally comprise a solid support and a metal cluster, and preferably comprise bonding between the metals of the metal cluster, and also bonding between the metal cluster and the solid support. Thus, the metal atom of the metal cluster can be bonded to at least one other metal atom of the metal cluster, preferably to six or fewer metal atoms of the metal cluster. The metal atom of the metal cluster can be bonded in addition to at least one hydrocarbon radical and/or to at least one hydrogen atom. The solid support can be chosen from a metal oxide, a refractory oxide, a molecular sieve, a zeolite, a metal phosphate and a material comprising a metal and oxygen, preferably silica. More particularly, the supported metal clusters can comprise tantalum (as a metal cluster) and silica (as a solid support), and preferably comprise bonding between tantalum and tantalum, and also bonding between tantalum and silica, in particular bonding between tantalum and oxygen on the silica surface. The catalyst (C2) can be chosen from the supported metal clusters described in International Patent Application WO 2006/060692, and prepared according to any methods described in said Application.

The catalyst (C2) is preferably chosen from metal hydrides, organometallic compounds and organometallic hydrides, particularly supported on, more specifically grafted onto, a solid support. It can comprise at least one metal chosen from scandium, yttrium, lanthanum, titanium, zirconium, hafnium, vanadium, niobium, tantalum, chromium, molybdenum, tungsten, rhenium, ruthenium, osmium, nickel, iridium, palladium, platinum, cerium and neodymium. The metal of the catalyst can be chosen, preferably, from yttrium, titanium, zirconium, hafnium, vanadium, niobium, tantalum, chromium, molybdenum, tungsten, rhenium, ruthenium and platinum, and more especially chosen from niobium, tantalum, molybdenum, tungsten and rhenium.

The catalyst (C2) can be chosen from metal hydrides, and preferably from organometallic compounds and organometallic hydrides, comprising more particularly at least one hydrocarbon radical (R), either saturated or unsaturated, linear or branched, having preferably from 1 to 20, more particularly from 1 to 14 carbon atoms. The hydrocarbon radicals (R) can be chosen from alkyl radicals, more particularly either saturated or unsaturated, linear or branched, aliphatic or alicyclic, for example alkyl, alkylidene or alkylidyne radicals, more particularly from C₁ to C₁₀, from aryl radicals, more particularly from C₆ to C₁₂, and from aralkyl, aralkylidene or aralkylidyne radicals, more particularly from C₇ to C₁₄. Thus, the metal atom of the catalyst (C2) is preferably bonded to at least one hydrogen atom and/or to at least one hydrocarbon radical (R), in particular by a single, double or triple carbon-metal bond.

The catalyst (C2) preferably selected-from metal hydrides, organometallic compounds and organometallic hydrides, can be furthermore modified by one or more ligands, such as “ancillary” ligands, preferably comprising at least one oxygen atom and/or at least one-nitrogen atom, and particularly chosen from oxo, alkoxo, aryloxo, aralkyloxo, nitrido, imido and amido ligands. Generally, by oxo, alkoxo, aryloxo, aralkyloxo, nitrido, imido and amido ligands are respectively meant:

a divalent oxo radical of general formula: ═O

a monovalent alkoxo, aryloxo or aralkyloxo radical of general formula: —OR′

a trivalent nitrido radical of general formula: ≡N

a divalent imido radical of general formula: ═NR″, and

a monovalent amido radical of general formula: —NR¹R²,

general formulae in which O represents an oxygen atom, N represents a nitrogen atom, R′ represents an hydrogen atom or a monovalent hydrocarbon radical selected respectively from alkyl, aryl and aralkyl radicals, R″ represents a n hydrogen atom or a monovalent hydrocarbon radical, and R¹ and R², being identical or different, represent an hydrogen atom or a monovalent hydrocarbon radical. More particularly, R′, R″, R¹ and R² can be a monovalent hydrocarbon radical, either saturated or unsaturated, linear or branched, comprising from 1 to 20, preferably from 1 to 14 carbon atoms, and particularly chosen from alkyl radicals, preferably from C₁ to C₁₀, aralkyl radicals, preferably from C₇ to C₁₄, and aryl radicals, preferably from C₆ to C₁₂.

Thus, when the catalyst (C2) chosen from metal hydrides, organometallic compounds and organometallic hydrides comprises at least one of the above-mentioned ligands, the metal of the catalyst is not only bonded to at least one hydrogen atom and/or to at least one hydrocarbon radical (R), but also to at least one of these ligands, e.g. to the O atom of the oxo radical by a double bond, or to the O atom of the alkoxo, aryloxo or aralkyloxo radical (OR′) by a single bond, or to the N atom of the nitrido radical by a triple bond, or to the N atom of the imido radical (NR″) by a double bond, or to the N atom of the amido radical (N¹R²) by a single bond. Generally, the presence of the oxo, alkoxo, aryl oxo, aralkyloxo, nitrido, imido and/or amido ligands in the catalyst (C2) favourably influences the behaviour of the catalyst in the simultaneous ethane self- and cross-metathesis reactions in stage (3).

In addition, the catalyst (C2) chosen from metal hydrides, organometallic compounds and organometallic hydrides is preferably supported on, or more particularly grafted onto, a solid support that can be chosen from metal or refractory oxides, sulphides, carbides, nitrides and salts, and from carbon, mesoporous materials, organic/inorganic hybrid materials, metals and molecular sieves. The solid support can be chosen from silicas, aluminas, silica-aluminas and aluminium silicates, either simple or modified by other metals, and from zeolites, clays, titanium oxide, cerium oxide, magnesium oxide, niobium oxide, tantalum oxide and zirconium oxide. It can be chosen, preferably, from silicas, aluminas, silica-aluminas and aluminium silicates, either simple or modified by other metals. The catalyst (C2) is preferably a metal hydride or an organometallic hydride grafted onto a solid support, more specifically chosen from niobium hydrides or organometallic niobium hydrides grafted onto silica, tantalum hydrides or organometallic tantalum hydrides grafted onto silica, molybdenum hydrides or organometallic molybdenum hydrides grafted onto silica, tungsten hydrides or organometallic tungsten hydrides grafted onto alumina, and rhenium hydrides or organometallic rhenium hydrides grafted onto silica.

The catalyst (C2) chosen from metal hydrides, organometallic compounds and organometallic hydrides can be in particular chosen from the metal catalysts described in International Patent Applications WO 98/02244, WO 03/061823 and WO 2004/089541, and prepared according to any of the methods described in said Applications.

Stage (3) comprises contacting the mixture (M1) obtained in stage (1) or preferably the ethane isolated in stage (2) with the catalyst (C2). Thus, the ethane is preferably used in stage (3) as the only or substantially the only starting alkane, e.g. in the form of a mixture of ethane with less than 1% by mole of one or more other alkanes preferably selected from methane and higher alkanes, particularly the (C₃₊), preferably the (C₃₋₆) alkanes as present in the natural gas. In this case, the ethane used alone or substantially alone as the starting material in stage (3) can be directly derived from the fractionation of the mixture (M1) in stage (2).

The ethane can also be preferably used in stage (3) in the form of a mixture of ethane, preferably in a major molar proportion, with one or more other higher alkanes particularly selected from the other higher alkanes as present in the natural gas, such as the (C₃₊), preferably the (C₃₋₆) alkanes, e.g. propane, butanes, pentanes and hexanes, and sometimes higher alkanes, such as heptanes and octanes. Thus, the mixture of ethane with one or more other higher alkanes may comprise from more than 50% to 99% by mole of ethane, preferably from 60% to 99% by mole of ethane, particularly from 75% to 99% by mole of ethane.

The ethane can also be preferably used in stage (3) in mixture or in combination with the mixture of the (C₂₊), preferably the (C₂₊) alkanes obtained by fractionation of the natural gas in a preliminary stage as previously described and from which the methane can be derived and isolated, and then used in stage (1). The mixture of the (C₂₊), preferably (C₂₋₆) alkanes which can be mixed or combined with the ethane or the mixture (M1) in stage (3), may generally comprise the (C₂₊), preferably the (C₂₋₆) alkanes of the natural gas, i.e. the C₂ to C₈, preferably the C₂ to C₇, particularly the C₂ to C₆ alkanes present in the natural gas, in molar ratios between themselves similar or identical to those of these alkanes in the natural gas. More particularly, in stage (3), the mixture of the (C₂₊), preferably the (C₂₋₆) alkanes and the mixture (M1) obtained in stage (1) or preferably the ethane isolated in stage (2) can be contacted either separately or preferably simultaneously, particularly in the form of a pre-mixing, with the catalyst (C2).

The ethane can also be preferably used in stage (3) in mixture or in combination with one or more (C₃₊), preferably (C₃₋₆) alkanes separated from the mixture (M1) in stage (2), and isolated either separately from the ethane or in the form of a mixing with the ethane. More particularly, in stage (3), the (C₃₊), preferably the (C₃₋₆) alkanes and the mixture (M1) obtained in stage (1) or preferably the ethane isolated in stage (2) can be contacted either separately or preferably simultaneously, particularly in the form of a pre-mixing, with the catalyst (C2).

The ethane can also be preferably used in stage (3) by combining the two previous methods: i.e. the ethane can be advantageously used in mixture or in combination, separately or simultaneously, with the above-mentioned mixture of the (C₂₊), preferably the (C₂₋₆) alkanes, and with the above-mentioned (C₃₊), preferably (C₃₋₆) alkanes.

All these various methods of using the ethane in mixture or in combination with other higher alkanes in stage (3) favourably develop the simultaneous ethane self- and cross-metathesis reactions and push towards an optimum production of the higher alkane mixture comprising the (C₃₊), preferably the (C₄₊), especially the (C₅₊) alkanes.

More particularly, in stage (3), it is observed that ethane can react with itself in contact with the catalyst (C2), and produce more particularly methane and propane by a self-metathesis reaction according to equation (10):

2C₂H₆→CH₄+C₃H₈ (10)

It is observed furthermore that stage (3) can also involve simultaneously a cross-metathesis reaction of ethane with the propane obtained previously, and produce especially methane and butanes according to equation (11):

C₂H₆+C₃H₈→CH₄+C₄H₁₀ (11)

It is also observed that stage (3) can in addition involve simultaneously other similar cross-metathesis reactions of ethane, in particular between ethane and successively higher alkanes having at least 4 carbon atoms, such as obtained by other preceding cross-metathesis reactions of ethane, and produce in particular methane and higher alkanes having one additional carbon atom compared with the higher alkanes involved in the reactions, for example according to equations (12) to (15):

C₂H₆+C₄H₁₀→CH₄+C₅H₁₂ (12)

C₂H₆+C₅H₁₂→CH₄+C₆H₁₄ (13)

C₂H₆+C₆H₁₄→CH₄+C₇H₁₆ (14)

C₂H₆+C₇H₁₆→CH₄+C₈H₁₈ (15)

It is thus found that in the present process, the methane has to be first converted into ethane in stage (1), prior to be converted into the higher alkane mixture, such as in stage (3). Thus, for the first time it clearly appears that stage (1) for converting methane into ethane is a crucial intermediate stage which allows ultimately for methane to be easily converted into the higher alkane mixture comprising the (C₃₊), preferably the (C₄₊), especially the (C₅₊) alkanes, and preferably isolated in the form of a liquid alkane mixture.

Furthermore, it is also found that in stage (3), the contacting with the catalyst (C2) is preferably performed simultaneously with the fractionation of the resulting mixture (M2), so as to separate and to isolate the higher alkane mixture comprising the (C₃₊), preferably the (C₄₊), especially the (C₅₊) alkanes, particularly as soon as these alkanes are formed. Thus, it surprisingly appears that performing simultaneously the contacting and the fractionation in stage (3) broadly prevents from relatively important production of by-products, such as ethane and methane, to the detriment of the desired higher alkane mixture comprising the (C₃₊), preferably the (C₄₊), especially the (C₅₊) alkanes. It is also greatly recommended to perform the contacting and the fractionation in stage (3) simultaneously and continuously.

It is also important to note that stage (3) can be carried out with a particularly high overall yield, particularly when the methane produced during the contacting in stage (3) is then separated and isolated from the mixture (M2) and then recycled into stage (1) for pursuing the non-oxidative methane coupling reaction and optionally the methane-olysis reactions.

Furthermore, additional cross-metathesis reactions between the (C₃₊), (C₄₊) or (C₅₊) alkanes and other (C₃₊), (C₄₊) or (C₅₊) alkanes can also occur in stage (3) and lead to substantial productions of subsequently higher alkanes.

The contacting of stage (3) generally leads to formation of a mixture (M2) comprising methane and the higher alkane mixture comprising the (C₃₊), preferably the (C₄₊), especially the (C₅₊) alkanes, and optionally unreacted ethane and where applicable propane and/or butanes, particularly when the higher alkane mixture respectively comprising the (C₄₊), or the (C₅₊) alkanes are preferably produced (instead of the higher alkane mixture comprising the (C₃₊) alkanes). Under the operating conditions of the contacting in stage (3), the mixture (M2) is generally obtained in gaseous form or in mixed gaseous/liquid form.

The contacting of stage (3) can advantageously be performed in conditions suited to promoting the development of the simultaneous ethane self- and cross-metathesis reactions such as described above, in particular towards an optimum production of the higher alkane mixture comprising the (C₃₊), preferably the (C₄₊), especially the (C₅₊) alkanes Thus, it can be performed at a temperature chosen in a range of from 20 to 400° C., preferably 50 to 350° C., particularly 70 to 300° C., more particularly 100 to 250° C. It can also be performed under an absolute total pressure chosen in a range of from 0.1 to 50 MPa, preferably 0.1 to 30 MPa, particularly 0.1 to 20 MPa It can also be performed in the presence of at least one inert agent, either liquid or gaseous, in particular of at least one inert gas chosen from nitrogen, helium and argon. It can also be performed in the presence of hydrogen, preferably the hydrogen separated from the mixture (M1) in stage (2), in particular under a partial pressure of hydrogen chosen in a range of from 0.0001 to 10 MPa, preferably from 0.001 to 1 MPa.

The contacting of stage (3) can be performed in various ways, in a reaction zone (Z2) which can be either distinct and separate from the reaction zone (Z1) in stage (1), or not. It can be performed either intermittently (or discontinuously), or preferably continuously. It is possible, for example, to add the ethane (or the mixture (M1)) and optionally other higher alkanes (e.g. the (C₂₊), the (C₂₋₆), the (C₃₊) or the (C₃₋₆) alkanes as previously described) to the catalyst (C2), or to add the catalyst (C2) to the ethane (or to the mixture (M1)) and optionally to the other higher alkanes, or else to add the ethane (or the mixture (M1)), optionally the other higher alkanes and the catalyst (C2) simultaneously into a reaction zone (Z2).

The reaction zone (Z2) can be of a type identical to or different from that of the reaction zone (Z1) of stage (1), as described above. Thus, the reaction zone (Z2) can comprise at least one reactor chosen from static reactors, recycling reactors and dynamic reactors. For example, the contacting of stage (3) can be performed in a static reactor containing fixed quantities of the ethane (or the mixture (M1)), optionally of the other higher alkanes and of the catalyst (C2). The contacting of stage (3) can also be performed in a recycling reactor into which at least one component of the mixture (M2), for example the methane, the higher alkane mixture comprising the (C₃₊), preferably the (C₄₊), especially the (C₅₊) alkanes, and optionally unreacted ethane and where applicable propane and/or butanes, can be recycled, preferably continuously, after it has been preferably separated from the mixture (M2) beforehand. The contacting of stage (3) can also be performed in a dynamic reactor containing the catalyst (C2), in the form of solid particles, or as a bed or a distillation packing, through which the ethane (or the mixture (M1)) and optionally the other higher alkanes can pass or circulate, preferably continuously.

In practice, the contacting of stage (3) can be performed in a reaction zone (Z2) comprising at least one reactor chosen from tubular (or multi-tubular) reactors, distillation/condensation column reactors, slurry reactors, fluidised bed reactors, mechanically agitated bed reactors, fluidised and mechanically agitated bed reactors, fixed bed reactors; moving bed reactors and circulating bed reactors. The catalyst (C2) is generally solid, and can optionally be mixed with an inert solid agent chosen in particular from silicas, aluminas, silica-aluminas and aluminium silicates, either simple or modified by other metals. The catalyst (C2) can be arranged inside the tubes of a tubular (or multi-tubular) reactor. It can also be arranged inside a distillation/condensation column reactor, wherein it is preferably a component of a distillation/condensation system functioning as both a catalyst and a distillation/condensation packing, i.e. a packing for a distillation/condensation column having both a distillation/condensation function (as in the fractionation of stage (3)) and a catalytic function (as in the contacting of stage (3)): for example, rings, saddles, balls, granulates, irregular shapes, granulates, sheets, tubes, spirals, packed in bags, as described in American Patent U.S. Pat. No. 4,242,530. The catalyst (C2) can form the bed of a fluidised and/or mechanically agitated bed, fixed bed, moving bed or circulating bed of said reactors. The ethane (or the mixture (M1)) and optionally the other higher alkanes can be introduced in the form of a stream, preferably continuously, into one of said reactors, and pass or circulate into the tubes or through the beds or the distillation/condensation packing of said reactors. In order to promote the development of the simultaneous ethane self- and cross-metathesis reactions towards an optimum production of the higher alkane mixture comprising the (C₃₊), preferably the (C₄₊), especially the (C₅₊) alkanes, the contacting of stage (3) can advantageously be performed by withdrawing said higher alkane mixture and/or the methane out of the reaction zone (Z2), preferably continuously.

In practice, the contacting in stage (3) can be performed:

(i) by continuously introducing the ethane (or the mixture (M1)) and optionally the other higher alkanes into the reaction zone (Z2) containing the catalyst (C2), so as to form continuously the mixture (M2) comprising the methane and the higher alkane mixture comprising the (C₃₊), preferably the (C₄₊), especially the (C₅₊) alkanes, and optionally unreacted ethane and where applicable propane and/or butanes, and preferably simultaneously

(ii) by continuously withdrawing at least one part of the mixture (M2) out of the reaction zone (Z2), so as to subject said part of the mixture (M2) to the fractionation of stage (3) for separating said higher alkane mixture, and preferably the methane from the rest of the mixture, and preferably to return said rest of the mixture, particularly the unreacted ethane and where applicable the propane and/or the butanes, and optionally the other non-separated part of the mixture (M2) into the reaction zone (Z2), in particular so as to continue the simultaneous ethane self- and cross-metathesis reactions.

Stage (3) also comprises fractionating the mixture (M2), so as to separate and to isolate the higher alkane mixture comprising the (C₃₊), preferably the (C₄₊), especially the (C₅₊) alkanes, preferably in liquid form, and particularly to separate and to isolate the methane which is recycled into stage (1). The fractionation of stage (3) can be performed in various ways, either intermittently (or discontinuously), or preferably continuously and particularly simultaneously with the contacting of stage (3). It can comprise one or more methods of fractionation of the mixture (M2), in particular chosen from any known methods or combinations of methods for fractionation of such a mixture, preferably from the following six types of fractionation:
fractionation by change of physical state, preferably by change of liquid/gaseous phase of the mixture, in particular by distillation or by condensation, particularly by partial condensation, preferably by means of distillation/condensation column or tower, or distillation/condensation column reactor,
fractionation by molecular filtration, preferably by means of semi-permeable and selective membrane,
fractionation by adsorption, preferably by means of molecular sieve or any other adsorbent,
fractionation by absorption, preferably by means of absorbing oil,
fractionation by cryogenic expansion, preferably by means of expansion turbine, and
fractionation by compression, preferably by means of gas compressor.

Any method that is useful in general for separating a mixture of the (C₃₊), preferably the (C₄₊), especially the (C₅₊) alkanes from methane and optionally ethane and where applicable propane and/or butanes, can be utilized in the fractionation of stage (3).

The fractionation of stage (3) thus makes it possible to separate and to isolate the higher alkane mixture comprising the (C₃₊), preferably the (C₄₊), especially the (C₅₊) alkanes, preferably in liquid form, from the mixture (M2). It can also permit the methane to be separated and isolated from the mixture (M2), and then preferably recycled into stage (1) for pursuing the non-oxidative methane coupling reaction and optionally the methane-olysis reactions. In addition, it can permit the unreacted ethane and/or where applicable the propane and/or the butanes to be separated and isolated from the mixture (M2), and then preferably to be recycled, in particular together or separately, into the contacting of stage (3) for pursuing the simultaneous ethane self- and cross-metathesis reactions.

The fractionation of stage (3) can be performed in a fractionation zone (F2) which is either distinct and separate from the reaction zone (Z2) wherein stage (3) is performed, or located in a part of said reaction zone (Z2), preferably in a distillation/condensation column reactor, as previously mentioned and described in American Patent U.S. Pat. No. 4,242,530. The fractionation of stage (3) may also be carried out in an integral part of the contacting of stage (3).

For instance, a distillation/condensation column reactor can contain the catalyst (C2) and be advantageously used both for the contacting and the fractionation of stage (3) performed simultaneously. More particularly, the catalyst (C2) can be arranged inside the distillation/condensation column reactor, as a component of a distillation/condensation system functioning both as a catalyst and a distillation/condensation packing. Advantageously, the contacting and the fractionation of stage (3) can be simultaneously performed in at least one distillation/condensation column reactor comprising both a reaction zone and a fractionation zone. In the reaction zone, the contacting of the ethane (or of the mixture (M1)) and optionally the other higher alkanes with the catalyst (C2) can be performed as in the contacting of stage (3), so as to form the mixture (M2) preferably in liquid/gaseous phase. Simultaneously, in the fraction zone, the separation of the higher alkane mixture comprising the (C₃₊), preferably the (C₄₊), especially the (C₅₊) alkanes from the mixture (M2) can be performed as in the fractionation of stage (3), preferably by distillation/condensation, so as to isolate said higher alkane mixture.

More generally, a fractionation of the mixture (M2) can be performed by change of the physical state, preferably of the liquid/gaseous phase of the mixture, particularly by distillation or by condensation (or liquefaction), more particularly by partial condensation (or partial liquefaction), e.g. in at least one distillation/condensation column or tower, or in a distillation/condensation column reactor. The fractionation can be performed by refrigeration or cryogenic processes, in particular by at least one cooling of the mixture (M2) to at least one temperature at which at least one component of the mixture condenses and the rest of the mixture remains in gaseous form.

Generally, a fractionation of the mixture (M2) can also be performed by molecular filtration, preferably by passing the mixture (M2) through at least one semi-permeable and selective membrane, so as to arrest and to isolate at least one component of the mixture and to allow the rest of the mixture to pass through.

Generally, a fractionation of the mixture (M2) can also be performed by adsorption, preferably by passing the mixture (M2) over at least one molecular sieve or any other adsorbent, so as to retain at least one component of the mixture and to allow the rest of the mixture to pass through, and then by subjecting the component(s) thus retained to at least one desorption step, preferably by the TSA or the PSA method, so as to isolate said component(s).

Generally, a fractionation of the mixture (M2) can also be performed by absorption, preferably by contacting the mixture (M2) with an absorbing oil, so as to soak up at least one component of the mixture, to form an oil/component mixture and to allow the rest of the mixture to be free and not absorbed, and then by subjecting said oil/component mixture to a temperature above the boiling point of the component(s), but below that of the oil, so as to separate and to isolate said component(s) from the oil.

Generally, a fractionation of the mixture (M2) can also be performed by cryogenic expansion, preferably by dropping the temperature of the mixture (M2) to a temperature so as to condense at least one component of the mixture while maintaining the rest of the mixture in unchanged form, e.g. by means of an expansion turbine which rapidly expands the mixture and causes a significant drop of the temperature.

Generally, a fractionation of the mixture (M2) can also be performed by compression, preferably by compressing the mixture (M2), e.g. by means of a gas compressor, so as to condense at least one component of the mixture, to separate and to isolate it from the rest of the gaseous mixture.

For example, a fractionation of the gaseous mixture (M2) can be performed by change of physical state, preferably of the liquid/gaseous phase of the mixture, e.g. in at least one distillation/condensation column or tower, or in a distillation/condensation column reactor, in particular by at least one cooling of the gaseous mixture to a temperature at which the higher alkane mixture comprising the (C₃₊), preferably the (C₄₊), especially the (C₅₊) alkanes condense, so as to condense said higher alkane mixture, to separate and to isolate it in liquid form from the rest of the gaseous mixture comprising the methane and optionally the unreacted ethane and where applicable the propane and/or the butanes.

For example, a fractionation of the gaseous mixture (M2) can also be performed by molecular filtration of the mixture, in particular by passing the gaseous mixture through at least one semi-permeable and selective membrane, so as to arrest and to isolate the higher alkane mixture comprising the (C₃₊), preferably the (C₄₊), especially the (C₅₊) alkanes and to allow the rest of the gaseous mixture comprising the methane and optionally the unreacted ethane and where applicable the propane and/or the butanes to pass through. The semi-permeable and selective membranes can be chosen from any membranes that are suitable for arresting and separating a mixture of the (C₃₊), preferably the (C₄₊), especially the (C₅₊) alkanes from methane, optionally ethane and where applicable propane and/or butanes. They can be preferably chosen from zeolite membranes, porous alumina membranes, ceramic membranes, flowing liquid membranes and supported liquid membranes.

For example, a fractionation of the gaseous mixture (M2) can also be performed by adsorption, in particular by passing the gaseous mixture through at least one molecular sieve or any other adsorbent, so as to retain the higher alkane mixture comprising the (C₃₊), preferably the (C₄₊), especially the (C₅₊) alkanes and to allow the rest of the gaseous mixture comprising the methane and optionally the unreacted ethane and where applicable the propane and/or the butanes to pass through, and then by subjecting said higher alkane mixture thus retained to at least one desorption step, for instance performed by the TSA or the PSA method, so as to isolate it. The adsorbents can be chosen from any adsorbents that are suitable for retaining and separating a mixture of the (C₃₊), preferably the (C₄₊), especially the (C₅₊) alkanes from methane, optionally ethane and where applicable propane and/or butanes. They can be preferably chosen from molecular sieves, silica gels, activated charcoals and activated carbons.

For example, a fractionation of the gaseous mixture (M2) can also be performed by absorption, preferably by contacting the gaseous mixture (M2) with an absorption oil, e.g. in an absorption tower, so as to soak up the higher alkane mixture comprising the (C₃₊), preferably the (C₄₊), especially the (C₅₊) alkanes aid to form an oil/higher alkanes mixture, and then preferably by subjecting said oil/higher alkanes mixture, e.g. in a recovery tower, to a temperature above the boiling point of said higher alkanes, but below that of the oil, so as to separate and to isolate said higher alkane mixture from the oil and from the rest of the gaseous mixture comprising the methane and optionally the unreacted ethane and where applicable the propane and/or the butanes.

For example, a fractionation of the gaseous mixture (M2) can also be performed by cryogenic expansion, preferably by dropping the temperature of the gaseous mixture (M2) in particular by use of external refrigerants for pre-cooling the gaseous mixture and then by means of an expansion turbine which rapidly expands the pre-cooled gaseous mixture and causes the temperature to drop significantly, so as to condense the higher alkane mixture comprising the (C₃₊), preferably the (C₄₊), especially the (C₅₊) alkanes, to separate and to isolate it in liquid form from the rest of the gaseous mixture comprising the methane and optionally the unreacted ethane and where applicable the propane and/or the butanes.

For example, a fractionation of the gaseous mixture (M2) can also be performed by compression, preferably by compressing the gaseous mixture (M2), e.g. by means of a gas compressor, so as to condense the higher alkane mixture comprising the (C₃₊), preferably the (C₄₊), the (C₅₊) alkanes, to separate and to isolate it in liquid form from the rest of the gaseous mixture comprising the methane and optionally the unreacted ethane and where applicable the propane and/or the butanes.

The fractionation in stage (3) can be performed advantageously by a double (or multiple) fractionation of the mixture: (M2), comprising a combination of two (or more) successive fractionations of an identical or different type, chosen from the six above-mentioned types of fractionation, so as in particular to separate and to isolate (a) the higher alkane mixture comprising the (C₃₊), preferably the (C₄₊), especially the (C₅₊) alkanes, (b) the methane and (c) unreacted ethane and where applicable propane and/or butanes.

Thus, the methane can be advantageously separated and isolated in the fractionation of stage (3), and then recycled into stage (1), particularly for pursuing the non-oxidative methane coupling reaction and optionally the methane-olysis reactions. In the same way, the unreacted ethane and/or where applicable the propane and/or the butanes is/are also preferably separated and optionally isolated in the fractionation of stage (3), in particular together or separately, and then preferably recycled into the contacting of stage (3), particularly for pursuing the simultaneous ethane self- and cross-metathesis reactions. The separation, isolation and recycling of the methane are particularly advantageous in the process of the invention, since the methane is produced in large quantities in the contacting of stage (3), and its recycling into stage (1) greatly increases the overall yield of the process. The same applies to the separation, isolation and recycling of the unreacted ethane and where applicable of the propane and/or the butanes, since it (or their) recycling into the contacting of stage (3) substantially increases the output of said stage.

For example, a double fractionation can be performed by subjecting the gaseous mixture (M2) comprising the methane, the higher alkane mixture comprising the (C₃₊), preferably the (C₄₊), especially the (C₅₊) alkanes, and unreacted ethane and where applicable propane and/or butanes, to a combination of two successive fractionations of one and the same type, namely by change of physical state, preferably of the liquid/gaseous phase of the mixture, e.g. in one or more distillation/condensation columns or towers, or in a distillation/condensation column reactor:

(i) by a first cooling of the gaseous mixture (M2) to a temperature at which the higher alkane mixture comprising the (C₃₊), preferably the (C₄₊), especially the (C₅₊) alkanes condenses, so as to condense said higher alkane mixture, and to separate and to isolate it in liquid form from the rest of the gaseous mixture comprising the methane, the unreacted ethane and where applicable the propane and/or the butanes, and

(ii) by a second cooling of said rest of the gaseous mixture to a temperature at which the ethane and where applicable the propane and/or the butanes condense(s), so as to condense the ethane and where applicable the propane and/or the butanes, and to separate and to isolate it (or them) in liquid form from the methane which is preferably isolated in its turn in gaseous form.

In practice, a double fractionation can also be performed by subjecting the above-mentioned gaseous mixture (M2) to a combination of two successive fractionations of one and the same type, namely by molecular filtration:

(i) by passing the gaseous mixture (M2) through a first semi-permeable and selective membrane, so as to arrest and to isolate the higher alkane mixture of the (C₃₊), preferably the (C₄₊), especially the (C₅₊) alkanes, and to allow the rest of the gaseous mixture comprising the methane, the unreacted ethane and where applicable the propane and/or the butanes to pass through, and

(ii) by passing said rest of the gaseous mixture through a second semi-permeable and selective membrane, so as to arrest and to isolate the ethane and where applicable the propane and/or the butanes, and to allow the methane to pass through and preferably to be isolated in its turn in gaseous phase.

In practice, a double fractionation can also be performed by subjecting the above-mentioned gaseous mixture (M2) to a combination of two successive fractionations of one and the same type, namely by adsorption:

(i) by passing the gaseous mixture (M2) onto a first molecular sieve or any other adsorbent, so as to retain the higher alkane mixture comprising the (C₃₊), preferably the (C₄₊), especially the (C₅₊) alkanes, and to allow the rest of the gaseous mixture comprising the methane, the unreacted ethane and where applicable the propane and/or the butanes to pass through, then

(ii) by passing said rest of the gaseous mixture onto a second molecular sieve or any other adsorbent, so as to retain the unreacted ethane and where applicable the propane and/or the butanes, and to allow the methane to pass through and preferably to be isolated in gaseous form, and

(iii) by respectively submitting said higher alkane mixture, the unreacted ethane and where applicable the propane and/or the butanes thus retained to desorption steps, in particular performed by the TSA or PSA method, so as respectively to isolate said higher alkane mixture, the unreacted ethane and where applicable the propane and/or the butanes.

In practice, it is also possible to perform a double fractionation by subjecting the above-mentioned gaseous mixture (M2) to a combination of two successive fractionations of one and the same type, namely by absorption, e.g. in one or more absorption towers:
(i) by contacting the gaseous mixture (M2) with a first absorbing oil, so as to soak up the higher alkane mixture of the (C₃₊), preferably the (C₄₊), especially the (C₅₊) alkanes to form an oil/higher alkanes mixture, and to allow the rest of the gaseous mixture comprising the methane, the unreacted ethane and where applicable the propane and/or the butanes to be free in gaseous form,
(ii) by contacting said rest of the gaseous mixture with a second absorbing oil, so as to soak up the unreacted ethane and where applicable the propane and/or the butanes, to form an oil/ethane/(propane and/or butanes) mixture, and to allow the methane to be free in gaseous form and preferably to be separated and isolated, and
(iii) by subjecting said oil/higher alkanes mixture to a temperature above the boiling point of said higher alkane mixture, but below that of the oil, e.g. in a recovery tower, so as to separate and to isolate said higher alkane mixture from the oil, and preferably by subjecting said oil/ethane/(propane and/or butanes) mixture to a temperature above the boiling point of ethane (and where applicable of propane and/or butanes), but below that of the oil, e.g. in a recovery tower, so as to separate and to isolate the unreacted ethane (and where applicable the propane and/or the butanes) from the oil.

In practice, it is also possible to perform a double fractionation by subjecting the above-mentioned gaseous mixture (M2) to a combination of two successive fractionations of one and the same type, namely by cryogenic expansion:
(i) by dropping the temperature of the gaseous mixture (M2) by means of an expansion turbine, so as to condense the higher alkane mixture comprising the (C₃₊), preferably the (C₄₊), especially the (C₅₊) alkanes, and to separate and to isolate it in liquid form from the rest of the gaseous mixture comprising the methane, the unreacted ethane and where applicable the propane and/or the butanes, and
(ii) by dropping the temperature of said rest of the gaseous mixture by means of an expansion turbine, so as to condense the unreacted ethane (and where applicable the propane and/or the butanes), to separate and to isolate it (or them) in liquid form from the methane which is preferably isolated in its turn in gaseous form.

In practice, it is also possible to perform a double fractionation by subjecting the above-mentioned gaseous mixture (M2) to a combination of two successive fractionations of one and the same type, namely by compression:
(i) by compressing the gaseous mixture (M2), e.g. by means of a gas compressor, so as to condense the higher alkane mixture comprising the (C₃₊), preferably the (C₄₊), especially the (C₅₊) alkanes, to separate and to isolate it in liquid form from the rest of the gaseous mixture comprising the methane, the unreacted ethane and where applicable the propane and/or the butanes, and
(ii) by compressing said rest of the gaseous mixture, e.g. by means of a gas compressor, so as to condense the unreacted ethane (and where applicable the propane and/or the butanes), to separate and to isolate it (or them) in liquid form from the methane which is preferably isolated in its turn in gaseous form.

It is also possible to perform a double (or multiple) fractionation by subjecting the above-mentioned gaseous mixture (M2) to a combination of two (or more) successive fractionations of a different type, chosen from the six above-mentioned types of fractionation.

A double fractionation of the above-mentioned gaseous mixture (M2) can, for example, be performed by combining two successive fractionations of a different type, namely:

the one by change of physical state, preferably of liquid/gaseous phase of the mixture, in particular by distillation or by condensation, and the other by molecular filtration, preferably by means of semi-permeable and selective membrane, or

the one by change of physical state, preferably of liquid/gaseous phase of the mixture, in particular by distillation or by condensation, and the other by adsorption, preferably by means of molecular sieve or any other adsorbent, or

the one by change of physical state, preferably of liquid/gaseous phase of the mixture, in particular by distillation or by condensation, and the other by absorption, preferably by means of absorbing oil, or

the one by change of physical state, preferably of liquid/gaseous phase of the mixture, in particular by distillation or by condensation, and the other by cryogenic expansion, preferably by means of expansion turbine, or

the one by change of physical state, preferably of liquid/gaseous phase of the mixture, in particular by distillation or by condensation, and the other by compression, preferably by means of gas compressor, or

the one by molecular filtration, preferably by means of semi-permeable and selective membrane, and the other by adsorption, preferably by means of molecular sieve or any other adsorbent, or

the one by molecular filtration, preferably by means of semi-permeable and selective membrane, and the other by absorption, preferably by means of absorbing oil, or

the one by molecular filtration, preferably by means of semi-permeable and selective membrane, and the other by cryogenic expansion, preferably by means of expansion turbine, or

the one by molecular filtration, preferably by means of semi-permeable and selective membrane, and the other by compression, preferably by means of gas compressor, or

the one by adsorption, preferably by means of molecular sieve or any other adsorbent, and the other by absorption, preferably by means of absorbing oil, or

the one by adsorption, preferably by means of molecular sieve or any other adsorbent, and the other by cryogenic expansion, preferably by means of expansion turbine, or

the one by adsorption, preferably by means of molecular sieve or any other adsorbent, and the other by compression, preferably by means of gas compressor, or

the one by absorption, preferably by means of absorbing oil, and the other by cryogenic expansion, preferably by means of expansion turbine, or

the one by absorption, preferably by-means of absorbing oil, and the other by compression, preferably by means of gas compressor, or

the one by cryogenic expansion, preferably by means of expansion turbine, and the other by compression, preferably by means of gas compressor.

Each of said combinations can be performed in a given chronological order or in a reverse order.

Thus, for example, the two successive fractionations of a different type can be performed advantageously:

(i) by cooling the gaseous mixture (M2) to a temperature at which the higher alkane mixture comprising the (C₃₊), preferably the (C₄₊), especially the (C₅₊) alkanes condenses, so as to condense said higher alkane mixture, to separate and to isolate it in liquid form from the rest of the gaseous mixture comprising the methane, the unreacted ethane and where applicable the propane and/or the butanes, and

(ii) by passing said rest of the gaseous mixture through a semi-permeable and selective membrane, so as to arrest and to isolate the unreacted ethane and where applicable the propane and/or the butanes, and to allow the methane to pass through and preferably to be isolated in its turn in gaseous form.

It is also possible to perform the two above-mentioned successive fractionations, but in a reverse order, that is to say:

(i) by passing the gaseous mixture (M2) through a semi-permeable and selective membrane, so as to arrest and to isolate the higher alkane mixture comprising the (C₃₊), preferably the (C₄₊), especially the (C₅₊) alkanes, and to allow the rest of the gaseous mixture comprising the methane, the unreacted ethane and where applicable the propane and/or the butanes to pass through, and

(ii) by cooling said rest of the gaseous mixture to a temperature at which the ethane and where applicable the propane and/or the butanes condense(s), so as to condense the unreacted ethane and where applicable the propane and/or the butanes, and to separate and to isolate it (or them) in liquid form from the methane which is preferably isolated in its turn in gaseous form.

Thus, e.g. by means of a double (or multiple) fractionation such as one of those described above, the methane can be advantageously isolated in the fractionation of stage (3) and preferably recycled into stage (1), particularly for continuing the non-oxidative methane coupling reaction and optionally the methane-olysis reactions. Furthermore, the unreacted ethane and where applicable the propane and/or the butanes is/are preferably isolated in the fractionation of stage (3), in particular together or separately, and advantageously is/are recycled into stage (3), particularly for pursuing the simultaneous ethane self- and cross-metathesis reactions.

The fractionation of stage (3) may also be performed as a simple compression step and as an integral part of the contacting of stage (3) through which the higher alkane mixture comprising the (C₃₊), preferably the (C₄₊), especially the (C₅₊) alkanes is separated e.g. by condensation.

The process of the invention has the advantage of converting methane easily with a high yield into the higher alkane mixture comprising the (C₃₊), preferably the (C₄₊), especially the (C₅₊) alkanes which is capable of being easily transportable in liquid form, under the standard temperature and pressure conditions or the conditions close to the latter. It also exhibits a very high “carbon atom efficiency” which can be equal to higher than 95%, even equal to higher than 99%. Furthermore, it can advantageously produce great amounts of hydrogen which can be isolated and used for supplying sufficient thermal and/or electrical energies for running the process.

The following examples illustrate the present invention.

### EXAMPLE 1

### Preparation of a Metal Catalyst (C1) Comprising an Organometallic Tantalum Hydride Grafted onto Silica

In a first stage, 1 g of a silica sold under the trade name “Aerosil 200”® by Degussa (Germany) was subjected to a dehydroxylation treatment under an absolute pressure of 10⁻² Pa, at 700° C., for 15 hours. At the end of this time, the silica thus treated was cooled to 25° C. under an argon atmosphere.

In a second stage, 1 g of the silica prepared beforehand was isolated and introduced into a reactor at 25° C. under an argon atmosphere. 350 mg of a precursor (Pr) in solution in 10 ml of pentane were then introduced into the reactor, the precursor (Pr) being tantalum tris(neopentyl)neopentylidene of general formula (16):

Ta[−CH₂−C(CH₃)₃]₃ [═CH—C(CH₃)₃] (16)

The reactor was then stirred for 2 hours, so as to form an organometallic tantalum compound grafted onto the silica. At the end of this time, the surplus of unreacted precursor (Pr) was eliminated by washings with pentane. The organometallic tantalum compound thus grafted onto the silica was isolated under argon. It contained 5.5 wt % of tantalum.

In a third stage, the organometallic tantalum compound grafted onto the silica prepared beforehand was subjected to a hydrogenolysis treatment by contacting with hydrogen, under an absolute hydrogen pressure of 73 kPa, at 150° C., for 15 hours. At the end of this time, a metal catalyst (C1) based on an organometallic tantalum hydride grafted onto silica and containing 5.5 wt % of tantalum was obtained and isolated under an argon atmosphere.

### EXAMPLE 2

### Preparation of a Catalyst (C2) Comprising an Organometallic Tungsten Hydride Grafted onto Alumina

In a first stage, 2 g of a γ-alumina (gamma-alumina) containing 90 wt % of alumina and 9 wt % of water, and sold by Johnson Matthey (Great Britain), were subjected to a calcination treatment under a dry air current at 500° C. for 15 hours, then to a dehydroxylation treatment under an absolute pressure of 10⁻² Pa, at 500° C., for 15 hours. At the end of this time, the alumina thus treated was cooled under an argon atmosphere to 25° C.

In a second stage, 2 g of the alumina prepared beforehand were isolated and introduced under an argon atmosphere into a reactor fitted with a magnetic agitator at 25° C. Then, 305 mg of a precursor (Pr′), namely tungsten tris(neopentyl)neo-pentylidyne of general formula (17):

W[−CH₂−C(CH₃)₃]₃ [≡C—C(CH₃)₃] (17)

were introduced.

The reactor was heated to 66° C. and the mixture thus obtained was agitated in the dry state for 4 hours. At the end of this time, the reactor was cooled to 25° C. under an argon atmosphere, and the solid mixture was then washed with n-pentane at 25° C. The solid compound thus washed was vacuum dried, then isolated under an argon atmosphere, so as to obtain an organometallic tungsten compound grafted onto alumina, containing 3.9 wt % of tungsten.

In a third stage, 40 mg of the organometallic tungsten compound grafted onto alumina prepared beforehand were isolated and subjected in a reactor to a hydro-genolysis treatment by contacting with hydrogen under an absolute hydrogen pressure of 73 kPa, at 150° C., for 15 hours. At the end of this time, the reactor was cooled to 25° C., and there was obtained and isolated under an argon atmosphere a metal catalyst (C2) based on an organometallic tungsten hydride grafted onto alumina, containing 3.9 wt % of tungsten.

### EXAMPLE 3

### Conversion of Methane into a Higher Alkane Mixture Comprising (C₄₊) Alkanes

According to stage (1), methane was introduced continuously into a reactor (R1) heated to 475° C., under an absolute total pressure of 5 MPa, and which contained the metal catalyst (C1) prepared in Example 1. By a non-oxidative methane coupling reaction, continuously there formed in the reactor (R1) a gaseous mixture (M1) comprising ethane, hydrogen and unreacted methane. The gaseous mixture (M1) was withdrawn continuously out of the reactor (R1).

According to stage (2), the gaseous mixture (M1) thus withdrawn was continuously introduced into a fractionation zone (F1) wherein ethane, hydrogen and unreacted methane were separated from each other continuously by change of physical state. The methane thus separated was isolated and recycled into the reactor (R1) wherein the methane coupling reaction was continued. During this time, the separated hydrogen was isolated in order to be used in part for activating and regenerating the catalysts (C1) and (C2), and the other main part as a fuel in hydrogen cells for producing thermal and electrical energies directly employed in the process.

According to stage (3), the ethane thus isolated was then continuously introduced into a reactor (R2) heated to 150° C., under a total absolute pressure of 2 MPa, and containing the catalyst (C2) prepared in Example 2, so as to be contacted with said catalyst. By simultaneous ethane self- and cross-metathesis reactions, continuously there formed in the reactor (R2) a gaseous mixture (M2) comprising the higher alkane mixture comprising the (C₄₊) alkanes, i.e. butanes, pentanes, hexanes, heptanes and octanes, and also methane, propane and unreacted ethane. The gaseous mixture (M2) was continuously withdrawn out of the reactor (R2). And simultaneously, the gaseous mixture (M2) thus withdrawn was continuously introduced into a fractionation zone (F2) by change of physical state, wherein the higher alkane mixture comprising the (C₄₊) alkanes was continuously separated, withdrawn and isolated in liquid form out of said zone (F2), while the methane and the unreacted ethane in mixture with the propane were also continuously separated, withdrawn and isolated out of said zone (F2). In the same time, the unreacted ethane in mixture with the propane thus isolated was continuously recycled into the reactor (R2) wherein the simultaneous ethane self- and cross-metathesis reactions were continued. During this time, the methane thus isolated was continuously recycled into the reactor (R1) wherein the non-oxidative methane coupling reaction was continued.

The higher alkane mixture was thus continuously isolated in liquid form, containing the C₄ to C₈ alkanes, completely free from impurities, with a high yield and with a particularly high “carbon atom efficiency” which was equal to 99%. All these results greatly resulted from the simultaneous operations of contacting and fractionating performed in stage (3). In addition, with the help of the hydrogen recovery, the total energy balance in the process was excellent.

### EXAMPLE 4

### Conversion of Natural Gas into a Higher Alkane Mixture Comprising (C₄₊) Alkanes

According to a purification stage, a natural gas was continuously subjected to a purification treatment for removing carbon dioxide and hydrogen sulphide.

According to a preliminary stage, the natural gas thus purified was introduced continuously into a fractionation zone by change of physical state, wherein the natural gas was cooled so that the alkanes (C₂₊), i.e. ethane, propane, butanes, pentanes and hexanes, condensed, and then were continuously separated and isolated in liquid form from the gaseous methane. Thus, the alkanes (C₂₊) were withdrawn and isolated continuously out of the fractionation zone, while the gaseous methane was also withdrawn and isolated continuously out of said zone.

According to stage (1), the methane thus isolated was continuously introduced into a reactor (R1) heated to 475° C., under an absolute pressure of 5 MPa, and containing the catalyst (C1) prepared in Example 1. By a non-oxidative methane coupling reaction, continuously there formed in the reactor (R1) a gaseous mixture (M1) comprising ethane, hydrogen and unreacted methane. The mixture (M1) was continuously withdrawn out of the reactor (R1).

According to stage (2), the mixture (M1) was continuously introduced into a fractionation zone (F1) by change of physical state, wherein the ethane and the unreacted methane were continuously separated from the hydrogen. The methane thus separated was continuously withdrawn and isolated out of said zone (F1), and then was continuously recycled into the reactor (R1) wherein the non-oxidative methane coupling reaction was continued. During this time, the hydrogen thus separated was also continuously withdrawn out of the fractionation zone (F1), and then was isolated in order to be used in part for activating and regenerating the catalysts (C1) and (C2), and the other main part as a fuel in hydrogen cells for producing thermal and electrical energies directly employed in the process.

According to stage (3), the ethane thus isolated in stage (2) and the (C₂₊) alkanes thus isolated in the preliminary stage were continuously introduced into a reactor (R2) heated to, 150° C., under an absolute pressure of 2 MPa, and containing the catalyst (C2) prepared in Example 2, so as to be contacted with said catalyst. In the reactor (R2), there formed by simultaneous ethane self- and cross-metathesis reactions a mixture (M2) comprising the higher alkane mixture comprising the (C₄₊) alkanes, i.e. butanes, pentanes, hexanes, heptanes and octanes, and also methane, propane and unreacted ethane. The mixture (M2) was continuously withdrawn out of the reactor (R2). And simultaneously, the mixture (M2) thus withdrawn was continuously introduced into a fractionation zone (F2) by change of physical state, wherein the higher alkane mixture comprising the (C₄₊) alkanes was continuously separated, withdrawn and isolated in liquid form out of said zone (F2), while the methane and the unreacted ethane in mixture with the propane were also continuously separated, withdrawn and isolated out of said zone (F2). In the same time, the unreacted ethane in mixture with the propane thus isolated were continuously recycled into the reactor (R2) wherein the simultaneous ethane self- and cross-metathesis reactions were continued. During this time, the methane thus isolated was continuously recycled into the reactor (R1) wherein the non-oxidative methane coupling reaction was continued.

The higher alkane mixture was thus continuously isolated in liquid form, containing the C₄ to C₈ alkanes, completely free from impurities, with. a high yield and with a particularly high “carbon atom efficiency” which was higher than 99%. All these results greatly resulted from the simultaneous operations of contacting and fractionating performed in stage (3). In addition, with the help of the hydrogen recovery, the total energy balance in the process was excellent.

### EXAMPLE 5

### Conversion of Natural Gas into a Higher Alkane Mixture Comprising (C₄₊) Alkanes

In a purification stage, a natural gas was continuously subjected to a purification treatment for removing carbon dioxide and hydrogen sulphide.

According to stage (1), the natural gas thus purified was continuously introduced into a reactor (R1) heated to 400° C., under an absolute pressure of 5 MPa, and containing the catalyst (C1) prepared in Example 1. In the reactor (R1), continuously there formed simultaneously by non-oxidative methane coupling reaction and methane-olysis reactions a gaseous mixture (M1) comprising ethane, hydrogen, unreacted methane and (C₃₊) alkanes (e.g. those of the natural gas, i.e. propane, butanes, pentanes and hexanes). The mixture (M1) was continuously withdrawn out of the reactor (R1).

According to stage (2), the mixture (M1) was then continuously introduced into a fractionation zone (F1) by change of physical state wherein the ethane in mixture with the (C₃₊) alkanes was continuously separated, withdrawn and isolated out of said zone (F1), while the methane and the hydrogen were also continuously separated, withdrawn and isolated out of said zone (F1). One part of the hydrogen thus isolated was used as an agent of activation and regeneration of the catalysts (C1) and (C2), and the other main part as a fuel in hydrogen cells for producing thermal and electrical energies directly employed in the process. The methane thus isolated was continuously recycled into the reactor (R1) wherein the non-oxidative methane coupling reaction and the methane-olysis reactions were simultaneously continued;

According to stage (3), the ethane in mixture with the (C₃₊) alkanes thus isolated was continuously introduced into a reactor (R2) heated to 150° C., under an absolute pressure of 2 MPa, and containing the catalyst (C2) prepared in Example 2, so as to be contacted with said catalyst. In the reactor (R2), continuously there formed by simultaneous ethane self- and cross-metathesis reactions a mixture (M2) comprising the higher alkane mixture comprising the (C₄₊) alkanes, i.e. butanes, pentanes, hexanes, heptanes, octanes, nonanes and decanes, and also methane, unreacted ethane and propane. The mixture (M2) was continuously withdrawn out of the reactor (R2). And simultaneously, the mixture (M2) thus withdrawn was continuously introduced into a fractionation zone (F2) by change of physical state, wherein the higher alkane mixture comprising the (C₄₊) alkanes was continuously separated, withdrawn and isolated in liquid form out of said zone (F2), while the methane and the ethane in mixture with the propane were also continuously separated, withdrawn and isolated out of said zone (F2). During this time, the methane thus isolated was continuously recycled into the reactor (R1) wherein the non-oxidative methane coupling reaction and the methane-olysis reactions were simultaneously continued. In the same time, the ethane in mixture with the propane thus isolated was continuously recycled into the reactor (R2) wherein the simultaneous ethane self- and cross-metathesis reactions were continued.

The higher alkane mixture was thus continuously isolated in liquid form, containing the C₄ to C₁₀ alkanes, completely free from impurities, with a high yield and with a particularly high “carbon atom efficiency” which was greater than 99%. All these results greatly resulted from the simultaneous operations of contacting and fractionating performed in stage (3). In addition, with the help of the hydrogen recovery, the total energy balance in the process was excellent.

### EXAMPLE 6

### Conversion of Natural Gas into a Higher Alkane Mixture Comprising (C₅₊) Alkanes

The conversion was exactly performed as in Example 5, apart from stage (3) wherein the mixture (M2) was continuously into a fractionation zone (F2) by change of physical under continuous operating conditions so that a higher alkane mixture comprising (C₅₊) alkanes, i.e. pentanes, hexanes, heptanes, octanes, nonanes and decanes, was continuously separated, withdrawn and isolated in liquid form out of said zone (F2), while the methane and the ethane in mixture with propane and butanes were also continuously separated, withdrawn and isolated out of said zone (F2). During this time, the methane thus isolated was continuously recycled into the reactor (R1) wherein the non-oxidative methane coupling reaction and the methane-olysis reactions were simultaneously continued. In the same time, the ethane in mixture with the propane and the butanes thus isolated was continuously into the reactor (R2) wherein the simultaneous ethane self- and cross-metathesis reactions were continued.

The higher alkane mixture was thus continuously isolated in liquid form, containing the C₅ to C₁₀ alkanes, completely free from impurities, with a high yield and with a particularly high “carbon atom efficiency” which is equal to 99%. All these results greatly resulted from the simultaneous operations of contacting and fractionating performed in stage (3). In addition, with the help of the hydrogen recovery, the total energy balance in the process was, excellent.

1. Process for converting methane into a higher alkane mixture comprising (C₄+) alkanes having 4 carbon atoms and more, characterized in that it comprises the following stages:
a stage (1) comprising contacting methane with a metal catalyst (C1) capable of producing, in contact with alkane, reactions involving the splitting and recombining of carbon-carbon and/or carbon-hydrogen and/or carbon-metal bonds, so as to form by a nonoxidative methane coupling reaction a mixture (M1) comprising ethane and hydrogen,
a stage (2) comprising fractionating the mixture (M1), so as to separate and to isolate the ethane, and
a stage (3) comprising simultaneously:
(i) contacting the ethane isolated in stage (2) with a metal catalyst (C2) capable of producing, in contact with alkane, reactions involving the splitting and recombining of carbon-carbon and/or carbon-hydrogen and/or carbon-metal bonds, said catalyst being identical to or different from the catalyst (C1), so as to form by simultaneous ethane self- and cross-metathesis reactions a mixture (M2) comprising methane and the higher alkane mixture comprising the (C₄+) alkanes, and
(ii) fractionating the mixture (M2) so as to separate and to isolate said higher alkane mixture.

2. Process according to claim 1, characterized in that the higher alkane mixture comprises (C₅+) alkanes having 5 carbon atoms and more.

3. Process according to claim 1, characterized in that the fractionating in stage (3) is performed in addition so as to separate and to isolate the methane which is recycled into stage (1).

4. Process according to claim 1, characterized in that all the stages are performed continuously.

5. Process according to claim 1, characterized in that in stage (1), the methane is used in the form of natural gas, preferably submitted to a purification stage prior to stage (1), in particular for removing one or more poisons capable of deactivating the metal catalysts (C1) and/or (C2), and/or one or more impurities.

6. Process according to claim 5, characterized in that the mixture (M1) obtained in stage (1) in addition comprises one or more (C₃₊) alkanes having 3 carbon atoms and more, preferably (C₃₋₆) alkanes.

7. Process according to claim 1, characterized in that prior to stage (1), a preliminary stage comprises separating and isolating by fractionation the methane from natural gas and preferably the rest of said natural gas comprising a mixture of (C₂₊) alkanes having 2 carbon atoms and more, particularly (C₂₋₆) alkanes which are preferably used in mixture or in combination with the ethane in the fractionating of stage (3).

8. Process according to claim 1, characterized in that the catalysts (C1) and (C2) are chosen from catalysts capable of alkane metathesis, preferably chosen from supported metal clusters, and especially from metal hydrides, organometallic compounds and organometallic hydrides, particularly supported on, more specifically grafted onto, a solid support.

9. Process according to claim 8, characterized in that the supported metal clusters comprise one or more metals of Groups 4, 5 and/or 6 of the Periodic Table of the Elements, preferably tantalum.

10. Process according to claim 8, characterized in that the catalysts chosen from metal hydrides, organometallic compounds and organometallic hydrides comprise at least one metal chosen from lanthanides, actinides and metals of Groups 2 to 12, preferably transition metals of Groups 3 to 12 of the Periodic Table of the Elements.

11. Process according to claim 8, characterized in that the catalysts comprise at least one metal chosen from scandium, yttrium, lanthanum, titanium, zirconium, hafnium, vanadium, niobium, tantalum, chromium, molybdenum, tungsten, rhenium, ruthenium, osmium, nickel, iridium, palladium, platinum, cerium and neodymium, preferably from yttrium, titanium, zirconium, hafnium, vanadium, niobium, tantalum, chromium, molybdenum, tungsten, rhenium, ruthenium and platinum, particularly from niobium, tantalum molybdenum, tungsten and rhenium.

12. Process according to claim 8, characterized in that the catalysts comprise a solid support chosen from metal or refractory oxides, sulfides, carbides, nitrides and salts, and from carbon, mesoporous materials, organic/inorganic hybrid materials, metals and molecular sieves.

13. Process according to claim 1, characterized in that stage (1) is performed at a temperature chosen in a range of from 50 to 700° C., preferably 150 to 700° C., particularly 200 to 650° C., in particular 100 to 600° C., especially 300 to 600° C.

14. Process according to claim 1, characterized in that stage (1) is performed under an absolute total pressure chosen in a range of from 0.1 to 100 MPa, preferably 0.1 to 50 MPa, in particular 0.1 to 30 MPa, especially 0.1 to 20 MPa.

15. Process according to claim 1, characterized in that in stage (2), the hydrogen is separated and isolated from the mixture (M1) and preferably used in one or more applications or stages, distinct or not from the present process, particularly used as an agent of activation or regeneration of the catalyst(s) (C1) and/or (C2), or as a fuel for producing thermal and/or electrical energies, in particular as a fuel in hydrogen fuel cells for producing electrical energy, energies being preferably used for running the process, or as a fuel for automobile, or as a reagent in a chemical, petrochemical or refinery plant.

16. Process according to claim 1, characterized in that in stage (2), the mixture (M1) in addition comprises unreacted methane which is separated and isolated, and then recycled into stage (1).

17. Process according to claim 6, characterized in that stage (2) comprises fractionating the mixture (M1) in addition so as to separate and to isolate one or more (C₃₊), preferably the (C₃₋₆) alkanes which are preferably used in mixture or in combination with the ethane in the contacting of stage (3).

18. Process according to claim 1, characterized in that stage (3) is performed at a temperature chosen in a range of from 20 to 400° C., preferably 50 to 350° C., in particular 70 to 300° C., especially 100 to 250° C.

19. Process according to claim 1, characterized in that stage (3) is performed under an absolute total pressure chosen in a range of from 0.1 to 50 MPa, preferably 0.1 to 30 MPa, in particular 0.1 to 20 MPa.

20. Process according to claim 1, characterized in that the mixture (M2) in addition comprises unreacted ethane which is separated and isolated in the fractionating of stage (3) and recycled into the contacting of stage (3).

21. Process according to claim 1, characterized in that the mixture (M2) in addition comprises propane which is separated and isolated preferably in mixture with unreacted ethane in the fractionating of stage (3) and recycled into the contacting of stage (3).

22. Process according, to claim 2, characterized in that the mixture (M2) in addition comprises propane and butanes which are separated and isolated preferably in mixture with unreacted ethane in the fractionating of stage (3) and recycled into the contacting of stage (3).
